(19) United States
(12) Patent Application Publication
(10) Pub. No.: US 2022/0204911 A1
(43) Pub. Date: Jun. 30, 2022

(54) FERROMAGNETIC MATERIAL WITH REMOTE RESPONSIVE CONTROL FOR DYNAMIC CELL CULTURE

(71) Applicant: The University of Kansas, Lawrence, KS (US)

(72) Inventors: Bryce Stottlemire, Lawrence, KS (US); Mei He, Gainesville, FL (US); Cory Berkland, Lawrence, KS (US)

(21) Appl. No.: 17/560,870

(22) Filed: Dec. 23, 2021

Related U.S. Application Data
(60) Provisional application No. 63/130,596, filed on Dec. 24, 2020.
(60) Provisional application No. 63/170,298, filed on Apr. 2, 2021.

(51) Int. Cl. C12M 1/42; C12N 5/0775; C12M 1/12; C12M 1/32

(52) U.S. Cl. CPC C12M 35/06; C12N 5/0667; C12M 25/14; C12N 2533/56; C12N 5/0665; C12M 23/12; C12N 2513/00; C12M 35/02

(57) ABSTRACT

Described herein is a system to remote-control magnetic actuation of dynamic cell culture. The systems described herein can include a porous, magnetic, elastomeric construct. The porous, magnetic, elastomeric construct can be formed from a composite including a biocompatible elastomer and a population of magnetic particles dispersed within the biocompatible elastomer.

### CROSS-REFERENCE TO RELATED APPLICATIONS

The application claims the benefit of U.S. Provisional Application No. 63/130,596, filed Dec. 24, 2020; and U.S. Provisional Application No. 63/170,298, filed Apr. 2, 2021, which are hereby incorporated herein by reference in their entirety.

### STATEMENT REGARDING FEDERALLY SPONSORED RESEARCH OR DEVELOPMENT

This disclosure was made with Government Support under Grant No. 1R35GM133794 awarded by the National Institutes of Health. The Government has certain rights to this disclosure.

### BACKGROUND

It has been well established that cells, especially mesenchymal stem cells (MSCs), can sense their mechanical environment via focal adhesion molecules. Such sensation transmits the mechanical signal to the actin cytoskeleton, in turn, can drive lineage specification of MSCs. The discoveries of how the mechanical environment cells sense shape their behavior has led to the development of dynamic culturing systems that look at controlling stem cell fate for tissue engineering and regenerative applications along with looking at creating physiological systems to study disease progression and high throughput drug screening. However, in vitro studies look at multiple interactions of mechanical stimuli with microenvironmental stimuli have been limited due to the low experimental throughput of current bioreactor systems. Current researchers have looked at developing microlevel dynamic culture systems that could overcome the limited capabilities of bioreactors given their bulky and complicated systems. These microlevel systems have consisted of piezo array systems and pneumatic pressure systems that provide an actuation force that is translated through a cell culture substrate but is limited by potential disturbances in force or vibration translation through the substrate. Therefore, it would be advantageous to develop materials that could be directly interfaced with the cell culture and deliver the desired stimulation. The compositions and methods disclosed herein address these and other needs.

### SUMMARY

Magnetic stimuli responsive materials are sensitive transformable materials that can provide programmable, predictive, and highly controllable dynamic ranges that have not been attainable via other field responsive mechanical metamaterials. Recent advancements using magnetic metamaterials for dynamic biological system platforms include magnetic substrate and magnetic pillar configurations to induce dynamic mechanical stimuli on 2D cultures or single cell analysis. These platforms shown promise in the potential of studying in vitro mechanotransduction with high spatial-temporal control with both complex and localized stimuli. The next frontier in the study of mechanobiology is developing novel 3D platforms that have ability to provide highly specific mechanical microenvironments that could be interfaced with 3D cell cultures to study cellular activity in 3D dynamic in vivo mimicking systems. The largest hurdle is creating a system that has both a high-fidelity 3D structure at the micron level and fast, tunable transformation potential.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIG. 1A-1D depicts the 3D Fabrication of smart ferromagnetic porous composites. (1A) The first step includes the printing of a 3D porous PVA template that represents the pores of the final magnetic composite structure. (1B) The PVA template is then placed in a pool of liquid ferromagnetic PDMS precursor and desiccated to allow for the magnetic precursor to penetrate the pores of the PVA structure via capillary action. The ferromagnetic PDMS filled PVA template (1C) is then baked to allow for the magnetic PDMS to cure. Lastly, the ferromagnetic PDMS filled template is placed in a hot water bath to leach and solubilize the PVA leaving only the (1D) magnetic PDMS composite.

FIG. 2A-2D demonstrates the resulting ferromagnetic porous composites following the 3D fabrication. (2A, 2B) Show macroscopic images of the resulting composites with (2A) rectilinear grid 90° offset and (2B) gyroid infill micropattern designs. (2C, 2D) Microscopic images are displayed to show the micropore structures of the (2C) rectilinear grid 90° offset and (2D) gyroid patterns.

FIG. 3 is a graph showing the mechanical analysis of the porous magnetic composites. Controlled compression (ElectroForce, TA Instruments) of carbonyl iron/PDMS composites (1:1 w/w) was used to find the Young's modulus of resulting magnetic composites fabricated via 70% infilled PVA template with 90-degree offset grid and gyroid patterns. The base to curing agent of the porous composites were also varied (10:1, 15:1, 20:1, w/w) to study the mechanical properties with different infill microstructures and crosslinking ratios.

FIG. 4A-4B shows the controlled deformation potential of the magnetic composites via an external magnetic field. (4A) Shows the gyroid infilled (20:1 base:cure) composite cylinders with no magnetic field (left) and an 500 mT magnetic field applied in the vertical (z) direction (right) immersed in PBS. (4B) Shows the gyroid (20:1 base:cure) reservoirs with a 5×5 mm cylinder void in the center with 3 mm porous walls. The reservoirs were placed under no magnetic field (left) and an applied magnetic field of 500 mT in the vertical (z) direction (right) with air as the medium.

FIG. 5A-5D exhibits the cell viability after 3D stem cell culture inside of ferromagnetic porous composites. Human adipose-derived mesenchymal stem cells (hASCs) were seeded in a 3D fibrin gel directly into the wells of the porous constructs, both with magnetic fillers (5C) and without (5D). Fibrinogen (10 mg/mL) and cells suspended in thrombin (2.5 U/mL) were pipetted into the composite wells and polymerized. The fibrin filled PDMS gels were cultured for 5 days in 48 well tissue culture plates, prior to removing the gels for viability studies. Gels were treated with calcein AM and ethidium homodimer solutions for 1 hour to stain live and dead cells. Preliminary studies indicate no difference in cell viability between cells cultured within magnetic composites (5A) and within PDMS-only composites (5B). Fluorescence microscopy was used to image stained cells, and the red and green channels were overlaid. Green fluorescence indicates live cells while red indicates dead cells. Scale bar=100 μm.

FIG. 6 indicates the waste and nutrient transport via the micropores of the composite well reservoirs. This shows the cumulative mass transport over time of an injected 3% percent alginate sol (15 mg/mL aniline blue) into a carbonyl iron/PDMS composite reservoir with a 5×5 mm cylinder well and 3 mm thick, porous walls. The relative cumulative release was found by measuring the absorbance (625 nm) of the PBS buffer outside the reservoir as a function of time.

FIG. 7A-7B (7A) illustrates sample 1 for the remote-control magnetic actuation dynamic cell culture system. A (7B) porous magnetic composite [1] (200) can be used to interface a 3D cell-laden gel [2] (500) 1 with contact with the bottom of the gel. The magnetic porous composite can be in contact and constrained by the bottom surface of a well plate well [3] (104).

FIG. 8A-8C shows sample 2 for the remote-control magnetic actuation dynamic cell culture system. (8A) A PDMS only porous composite (400) and a (8B) porous magnetic composite (200) can be used to interface a 3D cell-laden gel (500). (8C) The full system is displayed with the PDMS only porous composite [4] (400) in contact with the bottom the 3D gel [2] (500), and a [1] PDMS porous magnetic composite (200) can be in contact on the top of the 3D gel (500). The PDMS only composite (400) can be in contact and constrained by the bottom surface of a [3] well plate well (104). The 3D gel (500) can be placed in the dynamic cell culture system on top of the PDMS only porous composite (400). The magnetic composite (200) can be in contact with the surrounding walls but is free to move in the z-direction.

FIG. 9 shows the dynamic actuation of [2] the magnetic porous composite to induce a pressure on the 3D gel. A COMSOL simulation (COMSOL Multiphysics® Version 5.4) was performed to portray the potential for a magnetic composite to perform a deformation induced pressure to a 3D cell-laden gel. When an external magnetic field is applied to the magnetic composite, the magnetic particles induce a torque within the composite material that induces a deformation in the direction of the applied field. A COMSOL simulation using the Solid Mechanics Physics Module, was able to depict the deformation of the magnetic porous composite [1] on to the gel [2] with the bottom surface of the [4] PDMS only porous composite constrained to the surface of the well plate. Arbitrary values were used in the simulation to illustrate the pressure induced on the 3D gel with [5] representing the initial position of the magnetic porous composite before the application of the external magnetic field. It is to be observed that the gel is experiencing a pressure induced strain.

FIG. 10 represents sample 3 for the magnetic actuation dynamic cell culture system. The sample has the same set-up as sample 1 but allows for additional modifications to the interface surfaces. The PDMS only porous composite [4] can contact the bottom of the 3D gel [2], while the magnetic porous composite can contact the top of the gel like previously shown in the sample 1. However, the interface surfaces [6, 7] can be functionalized with soft lithography techniques or variants in infill percentages during the print to better control the contact surfaces applied on the 3D gel.

FIG. 11 shows sample 4 for the magnetic actuation dynamic cell culture system. This system can include of magnetic reservoir design that can have a magnetic reservoir design that can be in full contact with the 3D gel. The 3D printed magnetic reservoir can include a magnetic composite hollow inner cylinder design [8, 9] that can allow for the incorporation of a 3D gel to be injected directly into the hollow void space. The hollow cylinder design can be in the center of the magnetic reservoir [9] with the non-hollow composite design [10] to be on the top and bottom of the full magnetic reservoir design [11]. This allows for the injected 3D gel to be in full contact with the magnetic composite to allow for 360 degree actuation.

FIG. 12 shows sample 5 for the magnetic actuation dynamic cell culture system. Cells can be seeded on a magnetic porous composite, and the interconnected porosity of the magnetic composite can allow for the cells to migrate throughout the composite. Magnetic actuation then could be applied throughout the magnetic porous composite.

FIG. 13 illustrates the computer-aided design for the electromagnet nanovibration actuation device. The container is designed to hold a cell culture well plate in a defined location and induce location specific magnetic actuation depending on the location of the electromagnet in respect to the magnetic porous composite within the well plates. A circular compartment [13] is constructed to hold an electromagnet with the top surface of the electromagnet parallel to the surface of the container. Material is removed from the surface to fit the contours of a 96 well plate [14] and to ensure the wells are in contact with the surface of the electromagnet and defined in the x,y, and z axis. An aperture [15] is formed to run the electrical wires associated with the electromagnet out of the container. A power supply, relay, and microcontroller, outside the incubator, can be used to control the magnetic field strength, frequency, and duration.

FIG. 14A-14B shows a resulting 3D print of the computer-aided design for the electromagnet nanovibration actuation device. Displaying the 3D printed device without (14A) and with a 96-well plate (14B). The 3D Printed device 600A can enclose an electromagnet 301, microcontroller, and power supply with designed edges for the placement of vessel (100) such as a well plate.

FIG. 15A-15C displays the computer-aided design of the (15A) container 600B and (15B, 15C) stage 601B for the permanent magnet actuation system. The container 600B (15A) consists of two inner pillars [16] that are constructed to fit the contours of the two micro linear actuators. Material is removed from the top surface of the container to contain the well plate (103) in a defined location in the x, y, and z axis [14]. A stage (601B) is constructed (15B, 15C) to be attached to the linear actuators and move permanent magnets in both the positive and negative vertical direction. In the bottom surface of the stage (15B), gaps are formed to attach the stage to the stroke ends of the linear actuators [17]. On the top surface of the stage (15C), circular wells [18] are constructed to hold permanent magnets (302) at defined x and y locations. A power supply, multi-channel relay switch, and microcontroller will be used to control the position of the stage at a given time point. The frequency, amplitude, and duration of the dynamic movement of the stage will be defined by the parameters set for the microcontroller.

FIG. 16A-16B exhibits the 3D printed computer-aided design of the inner pillars, micro linear actuators, and the stage of the permanent magnetic actuation set-up. Shown are the (16A) lowest and (16B) highest vertical states of the linear actuated stage.

FIG. 17A-17B is an illustration of a 3D Printed container that can enclose an electromagnet, microcontroller, and power supply with designed edges for the placement of a well plate (103).

FIG. 18 is an illustration of the first construct including of a magnetic composite insert (200) at the bottom of a well (104) in a 96-well plate (103) and the 3D cell-laden gel (500) would be placed on top of the composite (200).

FIG. 19 is an illustration of the second construct including a PMDS composite insert (400) at the bottom of a well (104) in a 96-well plate (103), a 3D cell-laden gel (500) placed on top of the PMDS composite insert (400), and a non-constrained magnetic composite (200) placed above the cell laden gel (500).

FIG. 20A-20I is an illustration of using 3D printing and particulate leaching techniques to develop high-fidelity microporous magnetic composite constructs with various micro patterns and shapes.

FIG. 21 illustrates a simple interface system to have full control of the interface dynamics of a composite by contacting the top and bottom of a 3D cell matrix.

FIG. 22A-22I. 3D Fabrication of Smart Responsive Ferromagnetic Porous Composites. (22A-22C) Illustrates the process of fabricating magnetic porous composites utilizing both 3D printing and particulate leaching techniques. (22A) The first step includes 3D FDM printing a PVA sacrificial template which represents the pores of the final structure. (22B) The sacrificial template is placed in a magnetic Sylgard 184 (PDMS) precursor pool in a 1:1 w/w ratio (PDMS: Carbonyl Iron) and desiccated to allow for the precursor to fill the pores of the PVA sacrificial template. (22C) Magnetic PDMS precursor filled with PVA template is baked (65° C.) for PDMS curing. The PVA is leached out or dissolved away via a warm water bath. (22D-22F) Display images of resulting 3D constructs from the fabrication steps from A to C. scale bar=3 mm. (22G, 22I) Show microscopic brightfield images of 3D microstructures of the PVA sacrificial template and the PMDS- magnetic porous composite (22F). (22E) Shows the filament 3D printer used (N2, Raise 3D) with a 400 μm nozzle with submicron precision in the x-y axis.

FIG. 23A-23C. Investigation on the percentage of infill volumes (30%, 50%, 70%) along with the infill patterns (grid and gyroid) in PVA sacrificial template for influencing on resulting 3D constructs. (23A) Shows microscopic brightfield images of the microstructures of PVA templates and resulting magnetic-PDMS porous composites (23B). The scale bar is 400 μm. (23C) Macroscale images showing the overview of the magnetic-PDMS porous. The scale bar is 3 mm.

FIG. 24A-24D. Development of Complex Magnetic-PDMS Porous Composites with High Microlevel and Macrolevel Fidelity. The structures shown include (24A) cell culture well, (24B) reservoir on left with PDMS only reservoir on right shown to visualize the reservoir filled with an aniline blue dye hydrogel, (24C) hollow cylinder, (24D) KU logo.

FIG. 25A-25D. Mechanical and Remote Actuation Analysis of 3D Magnetic PDMS Porous Composites. (25A) Controlled compression analysis of 3D magnetic PDMS porous composites (1:1 w/w) under different base-to-cure cross linking ratios and microstructural patterns (mean±SD, n=5). The 3D magnetic PDMS porous composites were casted from 70% infilled PVA template with 90-degree offset grid and gyroid patterns. (25B-25C) The microscopic side view of the controlled deformation degree of 3D magnetic PDMS porous composites in (25B) grid pattern with 15:1 base: cure ratio and (25C) gyroid pattern with 20:1 base: cure ratio. The external magnetic field is applied consistently under different strengths. (25D) Quantitative strain data of both grid (15:1 base: cure ratio) and gyroid (20:1 base cure ratio) were obtained across a range of applied magnetic fields (mean±SD, n=5). The samples were submerged in PBS within 96-well plate shown in FIG. 29A for high-throughput 3D dynamic cell culture.

FIG. 26A-26C. Cell viability of 3D cultured human umbilical cord-derived mesenchymal stem cells (hUCMSCs) with 3D porous ferromagnetic interface. The hUCMSCs were seeded in a 3D fibrin gel directly on (26A) 3D magnetic-PDMS porous composites and (26B) PDMS only 3D porous constructs with different patterns (grid and gyroid) and base to curing agent ratios (10:1 and 15:1). A 3D fibrin gel was cultured on a well plate surface as a positive control. Live/dead staining was performed on the fourth day of cell culture (green-live; red-dead). (26C) Quantitative cell viability (n=3) was accessed using imaging processing software (Fiji, ImageJ). Scale bar=100 μm. Scale bar=100 μm.

FIG. 27A-27E. Characterization of 3D porous ferromagnetic interface for 3D Dynamic Culture of hMSCs. (27A) Analysis of the cumulative mass transport over time of an injected 3% percent alginate sol (15 mg/mL aniline blue) into a magnetic-PDMS composite reservoir with a 5×5 mm cylinder well and 3 mm thick porous walls. The relative cumulative release was measure on the absorbance (625 nm) of the PBS buffer outside the reservoir as a function of time. (27B) Fluorescence microscopic images showing the cell viability after 3D stem cell culture inside of a magnetic-PDMS porous well composites or PDMS only porous well (27C). Human adipose-derived mesenchymal stem cells (hADMSCs) were seeded in a 3D fibrin gel directly into the wells of both magnetic-PDMS (27D) and PDMS only (27E) well structures. Live/dead staining was performed on the fifth day of cell culture (green-live; red-dead). Scale bar=100 μm.

FIG. 28. Schematic illustration of three-step fabrication of 3D Porous Ferromagnetic Interface via combining 3D printing and particulate leaching methods (Top), which can produce versatile, precisely controlled microstructure patterns (bottom).

FIG. 29A-29C. 3D Printed Compartmental Boxes for Quantitative Strain Analysis and Dynamic Magnetic Actuation. (29A) 3D printed box was designed to accommodate a 96 well plate and have a location to insert a permanent magnet for magnetic actuation studies. An extension was connected to the 96 well plate box to hold a camera (iPhone XR) at a certain position and distance for reproducible image acquisition. (29B) The computer aided design (CAD) sketches of the 3D printed compartmental box is shown along with the permanent magnet spacers to apply different external magnetic field strengths to the magnetic composites in the 96 well plates. (29C) Displays a computer-aided design developed container and stage for a permanent magnet actuation system. The container consists of two inner pillars that are constructed to fit the contours of two micro linear actuators. Material is removed from the top surface of the container to contain the well plate in a defined location in the x, y, and z axis. A stage is constructed to be attached to the linear actuators and move permanent magnets in both the positive and negative vertical direction. In the bottom surface of the stage, gaps are formed to attach the stage to the stroke ends of the linear actuators. On the top surface of the stage, circular wells are constructed to hold permanent magnets at defined x and y locations. A power supply, multi-channel relay switch, and microcontroller will be used to control the position of the stage at a given time point. The frequency, amplitude, and duration of the dynamic movement of the stage will be defined by the parameters set for the microcontroller.

FIG. 30A-30D. Displays the recovery (30A, 30B) and precise actuation (30C, 30D) of magnetic porous composites for different dynamic actuation cycles. (30A-30B) Shows the capacity for both gyroid (30A) and grid (30B) porous composites to be able to reverse back to their original non-actuated height after the application of many different dyamic actuations. (30C-30D) Exhibits the assessment of the strain levels of porous magnetic (30C) gyroid and (30D) grid structures after different actuation cycles.

FIG. 31. Demonstrates a magnetic porous composite being deformed under dynamic magnetic field conditions. Fibrin hUCMSC laden 3D gels were polymerized on top of two gyroid (20:1) magnetic porous composites and images were obtained with and without the application of a 325 mT magnetic field.

FIG. 32A-32D. Porous Construct Groups for Cell Viability of Fibrin hUCMSCs Laden 3D Gels. Fibrin Gels were cultured on top of magnetic porous composites (32A-32B) and PDMS only porous composites (32C-32D). Two different construct infill patterns were investigated including grid (32A-32C) and gyroid (32B-32D) micropatterns.

FIG. 33 shows a permanent magnet box for the actuation platform. Two pillars encompass the linear actuators that attach to our permanent magnet stage. The linear actuators are able to move the stage freely along the z-axis with speeds around 0.59 inches a second. These linear actuators are then prompted by a microcontroller that controls their z direction translation, frequency, and duration. The box is also printed to allow for a cell culture well plate to be placed on top to allow for full external magnetic field potential and ability to visualize both the platform and the cell culture plate. The stage height and location of magnets can be modified to best suit the particular dynamic cell culture needs.

FIG. 34 show high-throughput actuation of a 96 well plate.

FIG. 35 shows material interface (contact variation) to overcome issues of PDMS membrane interfaces (pneumatic or electrode driven) such as bulky designs and lack of throughput, pneumatic or parabolic actuation (non uniaxial), complicated fabrication processes and difficult for implantation.

FIG. 36 illustrates system 1000 including a vessel (100); a porous, magnetic, elastomeric construct (200) sized to be positioned within the vessel (100); and a magnet (300) configured to apply a magnetic field within the construct (200) effective to deform the construct (200).

FIG. 37 illustrates system (1000A) including a vessel (100); a porous, magnetic, elastomeric construct (200) dimensioned to be positioned within the vessel (100) and in contact with the bottom surface (101) of the vessel (100); a 3D cell culture matrix (500) sized dimensioned to be positioned within the vessel (100) and disposed in contact with the porous, magnetic, elastomeric construct (200); and a magnet (300) configured to apply a magnetic field within the construct (200) effective to deform the construct (200).

FIG. 38 illustrates system (1000B) including a vessel (100); a porous, non-magnetic construct (400) dimensioned to be positioned within the vessel (100) and in contact with the bottom surface (101) of the vessel (100); a 3D cell culture matrix (500) sized dimensioned to be positioned within the vessel (100) and disposed in contact with the porous, non-magnetic, construct (400); a porous, magnetic, elastomeric construct (200) dimensioned to be positioned within the vessel (100) disposed in contact with the 3D cell culture matrix (500); and a magnet (300) configured to apply a magnetic field within the construct (200) effective to deform the construct (200).

FIG. 39 illustrates system (1000C) including a vessel (100); a porous, magnetic, elastomeric construct (200) dimensioned to be positioned within the vessel (100) and in contact with the bottom surface (101) of the vessel (100); a population of cells seeded (600) within the porous, magnetic, elastomeric construct (200); and a magnet (300) configured to apply a magnetic field within the construct effective to deform the construct (200).

Like reference symbols in the various drawings indicate like elements.

### DETAILED DESCRIPTION

A number of embodiments of the disclosure have been described. Nevertheless, it will be understood that various modifications may be made without departing from the spirit and scope of the invention. Accordingly, other embodiments are within the scope of the following claims.

### Definitions

To facilitate understanding of the disclosure set forth herein, a number of terms are defined below. Unless defined otherwise, all technical and scientific terms used herein generally have the same meaning as commonly understood by one of ordinary skill in the art to which this disclosure belongs.

To facilitate understanding of the disclosure set forth herein, a number of terms are defined below. Unless defined otherwise, all technical and scientific terms used herein have the same meanings as commonly understood by one of skill in the art to which the disclosed invention belongs. Publications cited herein and the materials for which they are cited are specifically incorporated by reference.

As used in this specification and the following claims, the terms “comprise” (as well as forms, derivatives, or variations thereof, such as “comprising” and “comprises”) and “include” (as well as forms, derivatives, or variations thereof, such as “including” and “includes”) are inclusive (i.e., open-ended) and do not exclude additional elements or steps. For example, the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof. Accordingly, these terms are intended to not only cover the recited element(s) or step(s), but may also include other elements or steps not expressly recited. Furthermore, as used herein, the use of the terms “a”, “an”, and “the” when used in conjunction with an element may mean “one,” but it is also consistent with the meaning of “one or more,” “at least one,” and “one or more than one.” Therefore, an element preceded by “a” or “an” does not, without more constraints, preclude the existence of additional identical elements.

The use of the term “about” applies to all numeric values, whether or not explicitly indicated. This term generally refers to a range of numbers that one of ordinary skill in the art would consider as a reasonable amount of deviation to the recited numeric values (i.e., having the equivalent function or result). For example, this term can be construed as including a deviation of ±10 percent of the given numeric value provided such a deviation does not alter the end function or result of the value. Therefore, a value of about 1% can be construed to be a range from 0.9% to 1.1%. Furthermore, a range may be construed to include the start and the end of the range. For example, a range of 10% to 20% (i.e., range of 10%-20%) can includes 10% and also includes 20%, and includes percentages in between 10% and 20%, unless explicitly stated otherwise herein.

It is understood that when combinations, subsets, groups, etc. of elements are disclosed (e.g., combinations of components in a composition, or combinations of steps in a method), that while specific reference of each of the various individual and collective combinations and permutations of these elements may not be explicitly disclosed, each is specifically contemplated and described herein.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

Reference will now be made in detail to specific aspects of the disclosed materials, compounds, compositions, articles, and methods, examples of which are illustrated in the accompanying Examples and Figures.

System

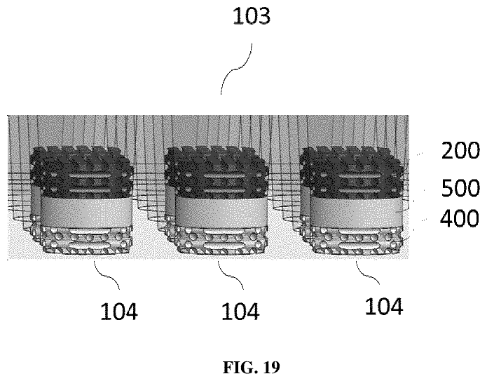

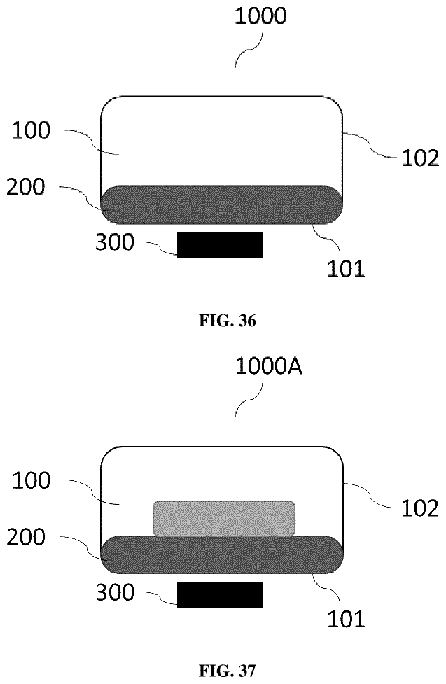

Described herein are systems for cell culture. In some embodiments, as shown in FIG. 36 the system (1000) can include a vessel (100); a porous, magnetic, elastomeric construct (200) sized to be positioned within the vessel (100); and a magnet (300) configured to apply a magnetic field within the construct (200) effective to deform the construct (200). In some embodiments, the vessel (100) can be substantially cylindrical and can include a bottom (101) and a side wall (102). In some embodiments, the system can include a vessel (100) that is a substantially cylindrical vessel (100) having a circular bottom surface (101) and a side wall (102). In some embodiments, the vessel (100) can include a well (104) of multiwell plate (103) having a circular bottom surface and a side wall as shown in FIGS. 18 and 19. In some embodiments, the system (1000) can include a substantially cylindrical porous, magnetic, elastomeric construct (201) dimensioned to be positioned within the vessel (100) and in contact with the bottom surface (101) of the vessel (100). In some embodiments, the system (1000) can further include a porous, non-magnetic construct (400) sized to be positioned within the vessel (100). In some embodiments, the system (1000) can include a porous, non-magnetic construct (400) dimensioned to be positioned within the vessel (100) and in contact with the bottom surface (101) of the vessel (100).

In some embodiments, the system (1000) can further include a 3D cell culture matrix (500) sized to be positioned within the vessel (100). In some embodiments, the system (1000) can include a 3D cell culture matrix (500) sized dimensioned to be positioned within the vessel (100) and disposed in contact with the porous, magnetic, elastomeric construct (200). In some embodiments, the system can include a 3D cell culture matrix (500) sized dimensioned to be positioned within the vessel (100) and disposed in contact with the porous, non-magnetic, construct (400). In some embodiments, the 3D cell culture matrix (500) can include a population of cells seeded (600) within a degradable polymer matrix.

In some embodiments, the polymer matrix can include, but is not limited to alginate, chitosan, agarose, fibrin, collagen, hyaluronic acid, a polyhydroxyalkanoate, a polyester, a polyalkylene oxide, a copolymer thereof, or a blend thereof. In some embodiments, the polymer matrix can include fibrin. In some embodiments, the polymer matrix can include alginate.

In some embodiments, as shown in FIG. 37 the system (1000A) can include a vessel (100); a porous, magnetic, elastomeric construct (200) dimensioned to be positioned within the vessel (100) and in contact with the bottom surface (101) of the vessel (100); a 3D cell culture matrix (500) sized dimensioned to be positioned within the vessel (100) and disposed in contact with the porous, magnetic, elastomeric construct (200); and a magnet (300) configured to apply a magnetic field within the construct (200) effective to deform the construct (200). In some embodiments, the system (1000A) can further include a porous, non-magnetic construct (400) dimensioned to be positioned within the vessel (100) and disposed in contact with the 3D cell culture matrix (500). In some embodiments, the system (1000A) can further include a second porous, magnetic, elastomeric construct (200a) dimensioned to be positioned within the vessel (100) and disposed in contact with the 3D cell culture matrix (500).

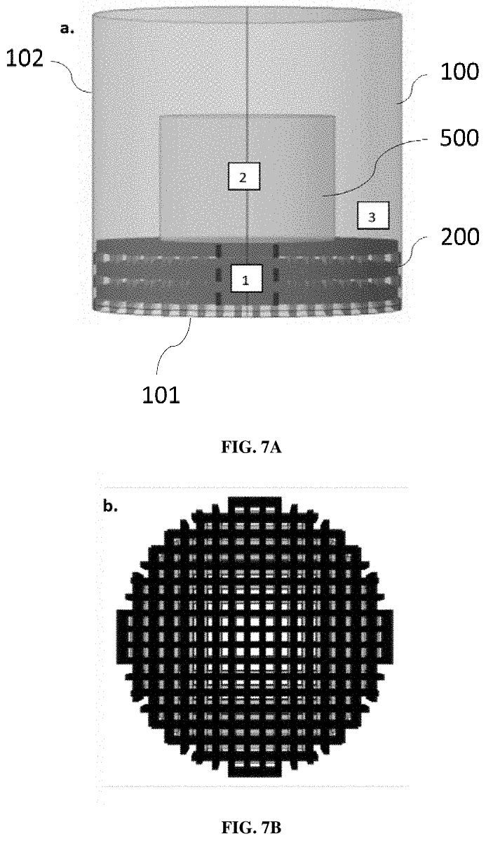

For example, in some embodiments, as shown in FIGS. 7A and 7B the system (1000A) can include a substantially cylindrical vessel (100) having a circular bottom surface (101) and a side wall (102); a substantially cylindrical porous, magnetic, elastomeric construct (200) dimensioned to be positioned within the vessel (100) and in contact with the bottom surface (101) of the vessel (100); a 3D cell culture matrix (500) sized dimensioned to be positioned within the vessel (100) and disposed in contact with the porous, magnetic, elastomeric construct (200); and a magnet (300) configured to apply a magnetic field within the construct (200) effective to deform the construct (200). In some embodiments, the system (1000A) can further include a substantially cylindrical porous, non-magnetic construct (400) dimensioned to be positioned within the vessel (100) and disposed in contact with the 3D cell culture matrix (500). In some embodiments, the system can further include a second substantially cylindrical porous, magnetic, elastomeric construct (200a) dimensioned to be positioned within the vessel (100) and disposed in contact with the 3D cell culture matrix (500).

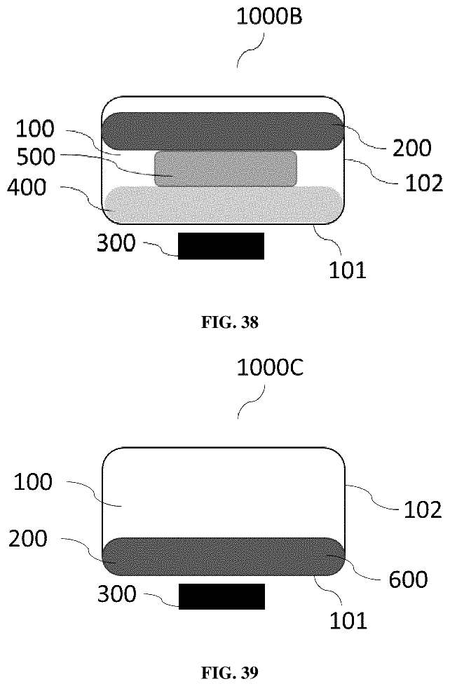

In some embodiments, as shown in FIG. 38 the system (1000B) can include a vessel (100); a porous, non-magnetic construct (400) dimensioned to be positioned within the vessel (100) and in contact with the bottom surface (101) of the vessel (100); a 3D cell culture matrix (500) sized dimensioned to be positioned within the vessel (100) and disposed in contact with the porous, non-magnetic, construct (400); a porous, magnetic, elastomeric construct (200) dimensioned to be positioned within the vessel (100) disposed in contact with the 3D cell culture matrix (500); and a magnet (300) configured to apply a magnetic field within the construct (200) effective to deform the construct (200).

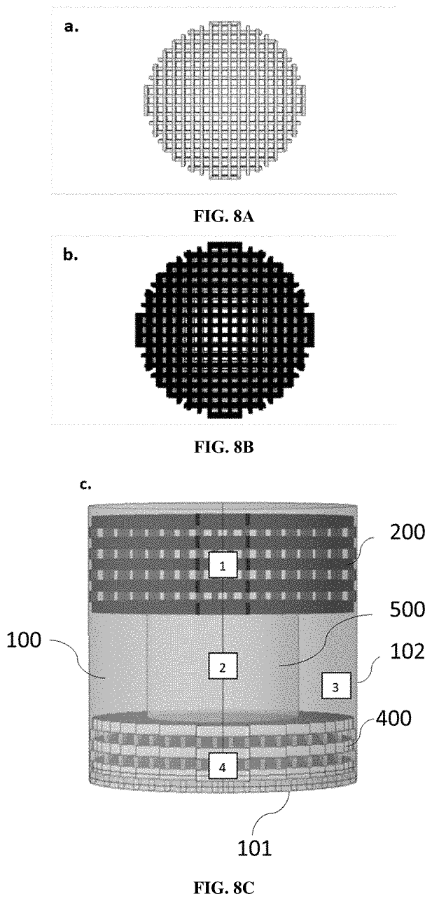

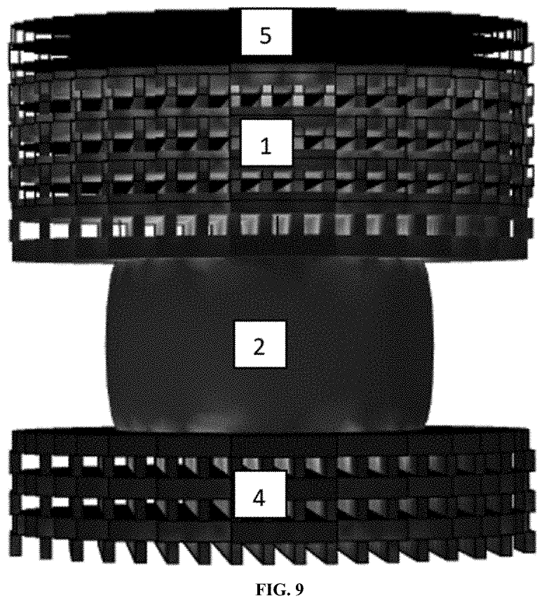

For example, in some embodiments, as shown in FIG. 8C the system (1000B) can include a substantially cylindrical vessel (100) having a circular bottom surface (101) and a side wall (102); a substantially cylindrical porous, non-magnetic construct (400) dimensioned to be positioned within the vessel (100) and in contact with the bottom surface (101) of the vessel (100); a 3D cell culture matrix (500) sized dimensioned to be positioned within the vessel (100) and disposed in contact with the substantially cylindrical porous, non-magnetic, construct (400); a substantially cylindrical porous, magnetic, elastomeric construct (200) dimensioned to be positioned within the vessel (100) disposed in contact with the 3D cell culture matrix (500); and a magnet (300) configured to apply a magnetic field within the construct (200) effective to deform the construct (200).

In some embodiments, as shown in FIG. 39 the system (1000C) can include a vessel (100); a porous, magnetic, elastomeric construct (200) dimensioned to be positioned within the vessel (100) and in contact with the bottom surface (101) of the vessel (100); a population of cells seeded (600) within the porous, magnetic, elastomeric construct (200); and a magnet (300) configured to apply a magnetic field within the construct effective to deform the construct (200). For example, in some embodiments, the system (1000C) can include a substantially cylindrical vessel (100) having a circular bottom surface (101) and a side wall (102); a substantially cylindrical porous, magnetic, elastomeric construct (200) dimensioned to be positioned within the vessel (100) and in contact with the bottom surface (101) of the vessel (100); a population of cells seeded (600) within the porous, magnetic, elastomeric construct (200); and a magnet (300) configured to apply a magnetic field within the construct effective to deform the construct.

In some embodiments, the system can further include a force sensor, a microcontroller, a power supply, or any combination thereof. In some embodiments, the system can further include a force sensor. In some embodiments, the system can further include a microcontroller. In some embodiments, the system can further include a power supply.

Described herein are also methods for remote responsive control of dynamic cell culture. The method can include operatively positioning a porous, magnetic, elastomeric construct in proximity to a 3D cell culture matrix; and applying a magnetic field to the porous, magnetic, elastomeric construct.

Porous, Magnetic, Elastomeric Constructs The systems described herein can include a porous, magnetic, elastomeric construct (200). The porous, magnetic, elastomeric construct can be formed from a composite including a biocompatible elastomer and a population of magnetic particles dispersed within the biocompatible elastomer. In some embodiments, the construct can have a substantially cylindrical shape.

In some embodiments, the construct can have a diameter of at least 60% of the inner diameter of the well, (e.g., at least 65%, at least 70%, at least 75%, at least 80%, at least 85%, at least 90%, or at least 95%). In some embodiments, the construct can have a diameter of 99% or less of the inner diameter of the well, (e.g., 95% or less, 90% or less, 85% or less, 80% or less, 75% or less, 70% or less, or 65% or less).

The construct can have a diameter ranging from any of the minimum values described above to any of the maximum values described above. For example, in some embodiments, the construct can have a diameter of from 60% to 99% of the inner diameter of the well (e.g., from 60% to 90%, from 60% to 80%, from 60% to 70%, from 70% to 90%, from 70% to 80%, from 80% to 90%, or from 85% to 95%).

In some embodiments, the porous, magnetic, elastomeric construct can have a porosity of at least 10%, (e.g., at least 15%, at least 20%, at least 25%, at least 30%, at least 35%, at least 40%, or at least 45%). In some embodiments, the porous, magnetic, elastomeric construct can have a porosity of 50% or less, (e.g., 45% or less, 40% or less, 35% or less, 30% or less, 25% or less, 20% or less, or 15% or less).

The porous, magnetic, elastomeric construct can have a porosity ranging from any of the minimum values described above to any of the maximum values described above. In some embodiments, the porous, magnetic, elastomeric construct can have a porosity of from 10% to 50%, (e.g., from 15% to 50%, from 20% to 50%, from 25% to 50%, from 30% to 50%, from 35% to 50%, from 40% to 50%, from 45% to 50%, from 15% to 40%, from 20% to 40%, from 25% to 40%, from 30% to 40%, from 35% to 40%, from 15% to 30%, from 20% to 30%, from 25% to 30%, from 15% to 20%, from 10% to 20%, from 10% to 30%, or from 10% to 40%).

In some embodiments, the porous, magnetic, elastomeric construct can be operatively configured to apply nanovibration, strain, or a combination thereof to a population of cells within the vessel. In some embodiments, the porous, magnetic, elastomeric construct can be operatively configured to apply nanovibration to a population of cells within the vessel. In some embodiments, the porous, magnetic, elastomeric construct can be operatively configured to apply strain, or a combination thereof to a population of cells within the vessel. In some embodiments, the porous, magnetic, elastomeric construct can be operatively configured to apply nanovibration and strain to a population of cells within the vessel.

In some embodiments, the population of cells can include but is not limited to cell able to convert mechanical cues into intracellular biochemical signals for driving cellular growth, differentiation, morphology, homeostasis, and disease. In some embodiments, the cells can include, but are not limited to induced pluripotent stem cells, adipose stem cells, bone marrow stem cells, synovium stem cells, dental pulp stem cells, neural stem cells, umbilical cord mesenchymal stem cells, chondrocytes, osteoblasts, myoblasts, fibroblasts, and myeloid cells.

Magnetic Particles

The magnetic particles can comprise any suitable population of magnetic particles. In some embodiments, the magnetic particles can comprise nanoparticles. The term “nanoparticle,” as used herein, generally refers to a particle of any shape having one or more dimensions ranging from 1 nm up to, but not including, 1 micron. In some embodiments, the nanoparticles can comprise a particle of any shape having one or more dimensions ranging from 1 nm up to, but not including, 1 micron; and one or more dimensions of 1 micron or more (e.g., from 1 micron to 10 microns, from 1 micron to 20 microns, from 1 micron to 25 microns, or from 1 micron to 50 microns).

In other embodiments, the magnetic particles can comprise microparticles. The microparticles can be of any shape, and have one or more dimensions ranging from 1 micron to 100 microns. In some embodiments, all dimensions can range from 1 micron to 100 microns.

In some embodiments, the population of magnetic particles is a monodisperse population of magnetic particles. In other embodiments, the population of magnetic particles is a polydisperse population of anisotropic magnetic particles. In some instances where the population of magnetic particles is monodisperse, greater than 50% of the particle size distribution, more preferably 60% of the particle size distribution, most preferably 75% of the particle size distribution lies within 10% of the median particle size.

The magnetic particles can comprise any suitable magnetic material, such as ferromagnetic alloys comprising iron, cobalt, nickel, or combinations thereof. some embodiments, the magnetic particles can include iron, cobalt, zinc, cadmium, nickel, gadolinium, chromium, copper, gold, silver, platinum, manganese, metal oxide, or an alloy thereof. In certain embodiments, the magnetic particles can include nickel particles.

In some embodiments, the magnetic particles can comprise anisotropic magnetic particles. Such particles can be formed using methods known in the art, including synthesis driven by appropriate shaping ligands, template-assisted synthesis, template-assisted electrodeposition, and magnetically directed assembly. Examples of such materials are described, for example, in Lisjak et al. “Anisotropic Magnetic Nanoparticles: A Review of their Properties, Synthesis, and Potential Applications,” Progress in Materials Science, 2018, 95; 286-328 (which is hereby incorporated by reference in its entirety for its description of anisotropic magnetic particles, and which is attached to this filing).

The magnetic particles can be essentially homogeneous throughout, meaning that the composition does not vary throughout the particle cross-section (from the particle surface to the particle center). Alternatively, the anisotropic particles can possess a non-homogeneous structure. For example, the particles may possess a core-shell structure, or a multilayer structure (e.g., a magnetic core coated by a non-magnetic shell material).

The magnetic particles may have any desired shape. In certain embodiments, the particles can have a non-spherical shape. As generally used herein, “non-spherical” is used to describe particles having at least one dimension differing from another dimension by a ratio of at least 1:1.10. In one embodiment, the non-spherical particles have at least one dimension which differs from another dimension by a ratio of at least 1:1.25. A wide variety of shapes are considered “non-spherical” shapes. For example, non-spherical particles may be in the shape of rectangular disks, high aspect ratio rectangular disks, rods, high aspect ratio rods, worms, oblate ellipses, prolate ellipses, elliptical disks, UFOs, circular disks, barrels, bullets, pills, pulleys, bi-convex lenses, ribbons, ravioli, flat pill, bicones, diamond disks, emarginated disks, elongated hexagonal disks, tacos, wrinkled prolate ellipsoids, wrinkled oblate ellipsoids, or porous elliptical disks.

In some embodiments, the magnetic particles can comprise rod-shaped particles. “Rod-shaped,” as used herein, refers to a particle which has an elongated spherical or cylindrical shape (e.g., the shape of a pill) or a flattened rod-shape, such as the shape of a green bean. The rod-shaped particles can have an aspect ratio of at least 1.25 (e.g., at least 1.5, at least 2, at least 2.5, or at least 5). “Aspect ratio,” as used herein, refers to the length divided by the diameter of a particle. In some embodiments, the rod-shaped particles can have an aspect ratio ranging from 1.25 to 95.

In some embodiments, the magnetic particles can be present in an amount of 20% by weight or less, based on the total weight of the construct, (e.g., 15% by weight or less, 10% by weight or less, 5% by weight or less, 1% by weight or less, or 0.5% by weight or less). In some embodiments, the magnetic particles can be present in an amount of 0.1% by weight or more, based on the total weight of the construct, (e.g., 0.5% by weight or more, 1% by weight or more, 5% by weight or more, 10% by weight or more, or 15% by weight or more).

The magnetic particles can be present in an amount ranging from any of the minimum values described above to any of the maximum values described above. For example, in some embodiments, the magnetic particles can be present in an amount of from 0.1% by weight to 20% by weight, based on the total weight of the construct, (e.g., from 0.1% by weight to 15% by weight, from 0.1% by weight to 10% by weight, from 0.1% by weight to 5% by weight, from 0.1% by weight to 2.5% by weight, from 0.1% by weight to 2% by weight, from 0.1% by weight to 1.5% by weight, from 0.1% by weight to 1% by weight, from 0.5% by weight to 15% by weight, from 0.5% by weight to 10% by weight, from 0.5% by weight to 5% by weight, from 0.5% by weight to 2.5% by weight, from 0.5% by weight to 2% by weight, from 0.5% by weight to 1.5% by weight, from 0.5% by weight to 1% by weight, from 1% by weight to 15% by weight, from 1% by weight to 10% by weight, from 1% by weight to 5% by weight, from 1% by weight to 2.5% by weight, from 1% by weight to 2% by weight, from 1% by weight to 1.5% by weight, from 2% by weight to 15% by weight, from 2% by weight to 10% by weight, from 2% by weight to 5% by weight, from 2% by weight to 2.5% by weight, from 5% by weight to 15% by weight, from 5% by weight to 10% by weight, or from 10% by weight to 15% by weight).

The magnetic particles can be present in the composition in an amount of from 0.01% by volume to 2.0% by volume (e.g., from 0.01% by volume to 1.5% by volume, from 0.01% by volume to 1.0% by volume, from 0.01% by volume to 0.75% by volume, from 0.01% by volume to 0.5% by volume, from 0.01% by volume to 0.2% by volume, or from 0.01% by volume to 0.15% by volume), based on the total volume of the composition.

In some embodiments, the magnetic particles can be uniformly dispersed throughout the elastomer. In other embodiments, the magnetic particles can by non-homogenously dispersed throughout the elastomer. For example, the magnetic particles can be at varying concentrations throughout the elastomer (e.g., at a higher concentration at a region of the construct in proximity to a magnet and at a lower concentration at a region further away from a magnet). In some embodiments, a gradient of magnetic particles can be dispersed within the elastomer.

Elastomers

The elastomer can include any suitable biocompatible elastomer. For example, the biocompatible elastomer can comprise an acrylonitrile butadiene styrene (ABS), polyphenylene sulfide (PPS), poly(meth)acrylate, polyphenylsulfone (PPSU), cyclic olefin copolymer (COC), polyetheretherketone (PEEK), polyurethane (PU), polyetherimide (PEI), polyphenylene ether (PPE), polycarbonate (PC), poly(ethyleneterephthalate glycol) (PETG), polysiloxane, or any combination thereof. In some embodiments, the biocompatible elastomer comprises a polysiloxane, such as polydimethylsiloxane (PDMS).

In some embodiments, the elastomer can include a polymer formed by curing of an elastomeric resin. The elastomeric resin can comprise an elastomeric resin suitable for use in an additive manufacturing process. Such materials are well known in the art. In some examples, the elastomeric resin can comprise a thermoplastic polymer such as acrylonitrile butadiene styrene (ABS), polyphenylene sulfide (PPS), polyphenylsulfone (PPSU), polyetheretherketone (PEEK), polyurethane (PU), polyetherimide (PEI), polyphenylene ether (PPE), polycarbonate (PC), and combinations thereof. In some embodiments, the elastomeric resin can comprise a crosslinkable composition (e.g., a blend of monomers, oligomers, and/or polymers which can be crosslinked during the additive manufacturing process). Depending on the additive manufacturing process employed, the crosslinkable composition can be selected such that crosslinking can be induced thermally and/or by impinging electromagnetic radiation (e.g., UV and/or visible light). In certain embodiments, the elastomeric resin can comprise a crosslinkable silicone composition. For example, the elastomeric resin can comprise (A) a first organosilicon compound having at least two ethylenically unsaturated moieties per molecule; and optionally (B) one or more additional organosilicon compounds. Suitable silicone compositions are known in the art. See, for example, U.S. Pat. No. 10,155,884 to Dow Silicones Corp., U.S. Patent Application Publication No. 2017/0312981 to Wacker Chemie AG, U.S. Patent Application Publication No. 2018/0370141 to Wacker Chemie AG, U.S. Patent Application Publication No. 2018/0066115 to Wacker Chemie AG, U.S. Patent Application Publication No. 2018/0186076 to Dow Corning Corp., and U.S. Patent Application Publication No. 2019/0100626 to Lawrence Livermore National Security LLC, each of which is hereby incorporated by reference in its entirety. Other suitable elastomeric resins are described, for example, in U.S. Patent Application Publication No. 20160319150 to Cornell University.

Other Components

Optionally, the porous, magnetic, elastomeric construct may include one or more additional components. For example, in some embodiments, the porous, magnetic, elastomeric construct may further include a non-magnetic filler. The non-magnetic filler may be, for example, an organic filler, an inorganic filler, a ceramic powder, or combinations thereof. The organic filler may be a polymer, such as, but not limited to, polystyrene, polyethylene, polypropylene, polysulfone, polyamide, polyimide, polyetheretherketone, etc. The organic filler can also be a smaller molecule either amorphous or crystalline in nature, and can be of in various shapes and sizes. The inorganic filler or ceramic powder can be any inorganic compounds that are compatible with the curing chemistry. Examples include, but are not limited to, silicon dioxide, titanium dioxide, zirconium dioxide, barium titanate, strontium titanate, etc. A mixture of more than one inorganic or organic with inorganic fillers are also suitable.

In some embodiments including the non-magnetic filler, the non-magnetic filler can be present as any suitable wt. % of the composition. In some embodiments, the non-magnetic filler can be present in an amount of 0.05 wt. % or more, 0.1 wt. % or more, 1 wt. % or more, 2 wt. % or more, 3 wt. % or more, 4 wt. % or more, 5 wt. % or more, 6 wt. % or more, 7 wt. % or more, 8 wt. % or more, 9 wt. % or more, 10 wt. % or more, 11 wt. % or more, 12 wt. % or more, 13 wt. % or more, 14 wt. % or more, 15 wt. % or more, 16 wt. % or more, 18 wt. % or more, 20 wt. % or more, 25 wt. % or more, 30 wt. % or more, 35 wt. % or more, 40 wt. % or more, 45 wt. % or more, 50 wt. % or more, 55 wt. % or more, 60 wt. % or more, 65 wt. % or more, 70 wt. % or more, 80 wt. % or more, or 85 wt. % or more. In some embodiments, the non-magnetic filler can be present in an amount of 90 wt. % or less, 85 wt. % or less, 80 wt. % or less, 75 wt. % or less, 70 wt. % or less, 65 wt. % or less, 60 wt. % or less, 55 wt. % or less, 50 wt. % or less, 45 wt. % or less, 40 wt. % or less, 35 wt. % or less, 30 wt. % or less, 25 wt. % or less, 20 wt. % or less, 15 wt. % or less, 10 wt. % or less, 5 wt. % or less, 1 wt. % or less, 0.5 wt. % or less, 0.1 wt. % or less, or 0.05 wt. % or less.

The non-magnetic filler can be present in an amount ranging from any of the minimum values described above to any of the maximum values described above. For example, in some embodiments, the non-magnetic filler can be present in an amount of from 0.01 wt. % to 90 wt. % of the compositions, (e.g., from 1 wt. % to 90 wt. %, from 5 wt. % to 90 wt. %, from 10 wt. % to 90 wt. %, from 15 wt. % to 90 wt. %, from 25 wt. % to 90 wt. %, from 30 wt. % to 90 wt. %, from 40 wt. % to 90 wt. %, from 50 wt. % to 90 wt. %, from 60 wt. % to 90 wt. %, from 70 wt. % to 90 wt. %, from 80 wt. % to 90 wt. %, from 1 wt. % to 80 wt. %, from 5 wt. % to 80 wt. %, from 10 wt. % to 80 wt. %, from 15 wt. % to 80 wt. %, from 25 wt. % to 80 wt. %, from 30 wt. % to 80 wt. %, from 40 wt. % to 80 wt. %, from 50 wt. % to 80 wt. %, from 60 wt. % to 80 wt. %, from 70 wt. % to 80 wt. %, from 1 wt. % to 75 wt. %, from 5 wt. % to 75 wt. %, from 10 wt. % to 75 wt. %, from 15 wt. % to 75 wt. %, from 25 wt. % to 75 wt. %, from 30 wt. % to 75 wt. %, from 40 wt. % to 75 wt. %, from 50 wt. % to 75 wt. %, from 60 wt. % to 75 wt. %, from 70 wt. % to 75 wt. %, from 55 wt. % to about 75 wt. %, from 1 wt. % to 60 wt. %, from 5 wt. % to 60 wt. %, from 10 wt. % to 60 wt. %, from 15 wt. % to 60 wt. %, from 25 wt. % to 60 wt. %, from 30 wt. % to 60 wt. %, from 40 wt. % to 60 wt. %, from 50 wt. % to 60 wt. %, from 1 wt. % to 50 wt. %, from 5 wt. % to 50 wt. %, from 10 wt. % to 50 wt. %, from 15 wt. % to 50 wt. %, from 25 wt. % to 50 wt. %, from 30 wt. % to 50 wt. %, from 40 wt. % to 50 wt. %, from 1 wt. % to 40 wt. %, from 5 wt. % to 40 wt. %, from 10 wt. % to 40 wt. %, from 15 wt. % to 40 wt. %, from 25 wt. % to 40 wt. %, from 30 wt. % to 40 wt. %, from 1 wt. % to 30 wt. %, from 5 wt. % to 30 wt. %, from 10 wt. % to 30 wt. %, from 15 wt. % to 30 wt. %, from 25 wt. % to 30 wt. %, from 1 wt. % to 20 wt. %, from 5 wt. % to 20 wt. %, from 10 wt. % to 20 wt. %, from 15 wt. % to 20 wt. %, from 1 wt. % to 10 wt. %, from 5 wt. % to 10 wt. %, from 1 wt. % to 5 wt. %, from 0.5 wt. % to 1 wt. %, from 0.5 wt. % to 5 wt. %, from 0.5 wt. % to 10 wt. %, from 0.5 wt. % to 20 wt. %, from 0.5 wt. % to 30 wt. %, from 0.5 wt. % to 40 wt. %, from 0.5 wt. % to 50 wt. %, from 0.5 wt. % to 60 wt. %, from 0.5 wt. % to 70 wt. %, from 0.5 wt. % to 80 wt. %, from 0.5 wt. % to 90 wt. %, from 0.05 wt. % to 1 wt. %, from 0.05 wt. % to 5 wt. %, from 0.05 wt. % to 10 wt. %, from 0.05 wt. % to 20 wt. %, from 0.05 wt. % to 30 wt. %, from 0.05 wt. % to 40 wt. %, from 0.05 wt. % to 50 wt. %, from 0.05 wt. % to 60 wt. %, from 0.05 wt. % to 70 wt. %, from 0.05 wt. % to 80 wt. %, from 0.05 wt. % to 90 wt. %).

The non-magnetic filler can have any suitable particle size, e.g., the longest dimension of the particle, such as the average longest dimension. In some embodiments, the non-magnetic filler can have a primary particle size of 100 microns or less, (e.g., 90 microns or less, 80 microns or less, 70 microns or less, 60 microns or less, 50 microns or less, 40 microns or less, 30 microns or less, 20 microns or less, 10 microns or less. In some embodiments, the non-magnetic filler can have a primary particle size of 5 microns or more, (e.g., 10 microns or more, 20 microns or more, 30 microns or more, 40 microns or more, 50 microns or more, 60 microns or more, 70 microns or more, or 80 microns or more).

The non-magnetic filler can have a primary particle size ranging from any of the minimum values described above to any of the maximum values described above. For example, the non-magnetic filler can have a primary particle size of from 5 microns to 100 microns, (e.g., 5 microns to 100 microns, 10 microns to 100 microns, 20 microns to 100 microns, 30 microns to 100 microns, 40 microns to 100 microns, 50 microns to 100 microns, 60 microns to 100 microns, 70 microns to 100 microns, from 80 microns to 100 microns, 90 microns to 100 microns, from 5 microns to 90 microns, from 10 microns to 90 microns, from 20 microns to 90 microns, from 30 microns to 90 microns, from 40 microns to 90 microns, from 50 microns to 90 microns, from 60 microns to 90 microns, from 70 microns to 90 microns, from 80 microns to 90 microns, from 5 microns to 80 microns, from 10 microns to 80 microns, from 20 microns to 80 microns, from 30 microns to 80 microns, from 40 microns to 80 microns, from 50 microns to 80 microns, from 60 microns to 80 microns, from 70 microns to 80 microns, from 5 microns to 70 microns, from 10 microns to 70 microns, from 30 microns to 70 microns, from 30 microns to 70 microns, from 40 microns to 70 microns, from 50 microns to 70 microns, from 60 microns to 70 microns, from 5 microns to 60 microns, from 10 microns to 60 microns, from 20 microns to 60 microns, from 30 microns to 60 microns, from 40 microns to 60 microns, from 50 microns to 60 microns, from 5 microns to 50 microns, from 10 microns to 50 microns, from 20 microns to 50 microns, from 30 microns to 50 microns, from 40 microns to 50 microns, from 5 microns to 40 microns, from 10 microns to 40 microns, from 20 microns to 40 microns, from 30 microns to 40 microns, from 5 microns to 30 microns, or from 10 microns to 30 microns, from 20 microns to 30 microns, from 5 microns to 20 microns, from 10 microns to 20 microns, from 5 microns to 10 microns. In some embodiments, the non-magnetic filler can have a primary particle size of 90 microns or more. In some embodiments, the non-magnetic filler can have a primary particle size of 5 microns or less. As used herein, “primary” particle size refers to the actual particles in their un-conglomerated state, which can optionally conglomerate to form larger “secondary” particles.

Examples of additional ingredients include, but are not limited to, adhesion promoters; dyes; pigments; anti-oxidants; initiators for crosslinking; carrier vehicles; heat stabilizers; flame retardants; thixotropic agents; flow control additives; inhibitors; extending and reinforcing fillers; and cross-linking agents.

One or more of the additives can be present as any suitable wt. % of the composition. In some embodiments, the additives can be present in an amount of 15 wt. % or less of the composition, (e.g., 14 wt. % or less, 13 wt. % or less, 12 wt. % or less, 11 wt. % or less, 10 wt. % or less, 9 wt. % or less, 8 wt. % or less, 7 wt. % or less, 6 wt. % or less, 5 wt. % or less, 4 wt. % or less, 3 wt. % or less, 2 wt. % or less, 1 wt. % or less, or 0.5 wt. % or less). In some embodiments, the additives can be present in an amount of 0.1 wt. % or more of the composition, (e.g., 0.5 wt. % or more, 1 wt. % or more, 2 wt. % or more, 3 wt. % or more, 4 wt. or more, 5 wt. % or more, 6 wt. % or more, 7 wt. % or more, 8 wt. % or more, 9 wt. % or more, 10 wt. % or more, 11 wt. % or more, 12 wt. % or more, 13 wt. % or more, or 14 wt. % or more).

The additives can be present in an amount ranging from any of the minimum values described above to any of the maximum values described above. For example, in some embodiments, the additives can be present in an amount ranging from 0.1 wt. % to 15 wt. % of the composition, (e.g., from 0.1 wt. % to 5 wt. %, from 0.1 wt. % to 10 wt. %, from 0.5 wt. % to 5 wt. %, from 0.5 wt. % to 10 wt. %, from 0.5 wt. % to 15 wt. %, from 1 wt. % to 5 wt. %, from 1 wt. % to 10 wt. %, from 1 wt. % to 15 wt. %, from 2 wt. % to 5 wt. %, from 2 wt. % to 10 wt. %, from 2 wt. % to 15 wt. %, from 5 wt. % to 10 wt. %, from 5 wt. % to 15 wt. %, from 8 wt. % to 10 wt. %, from 8 wt. % to 15 wt. %, from 10 wt. % to 15 wt. %).

Construct Fabrication

The porous, magnetic, elastomeric constructs described herein can be formed using an additive manufacturing process, such as fused deposition modeling (FDM), fused filament fabrication (FFF), fused pellet fabrication, fused particle fabrication, composite filament fabrication (CFF), direct ink writing (DIW), stereolithography (SLA), digital light processing, continuous liquid interface production, selective heat sintering, or selective laser sintering.

The constructs can be interfaced with a magnet (e.g., an electromagnet) configured to apply a magnetic field within the construct. By applying a magnetic field of varying strength, the mechanical properties of the construct (e.g., the Young's modulus of the material) can be varied and/or the construct can be deformed.

Magnet

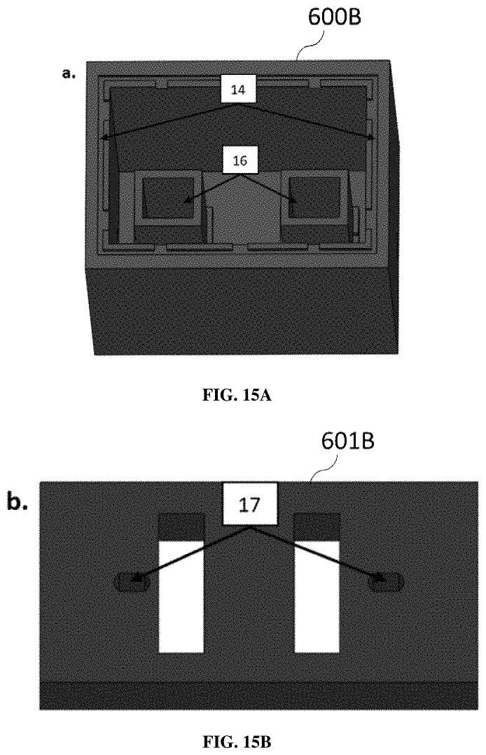

The systems described herein can include a magnet (300). The magnet can include a permanent magnet (302) as shown in FIG. 15A-15C. In some embodiments, the permanent magnet can generate an external magnetic field of at least 200 mT, (e.g., at least 250 mT, at least 300 mT, at least 350 mT, or at least 400 mT). In some embodiments, the permanent magnet can generate an external magnetic field of 450 mT or less, (e.g., 400 mT or less, 350 mT or less, 300 mT or less, or 250 mT or less).

The permanent magnet can generate an external magnetic field ranging from any of the minimum values described above to any of the maximum values described above. For example, in some embodiments, the permanent magnet can generate an external magnetic field of from 200 mT to 450 mT, (e.g., from 200 mT to 400 mT, from 250 mT to 300 mT, from 250 mT to 350 mT, from or from 250 mT to 400 mT, from 300 mT to 350 mT, from 300 mT to 400 mT, or from 350 mT to 400 mT).

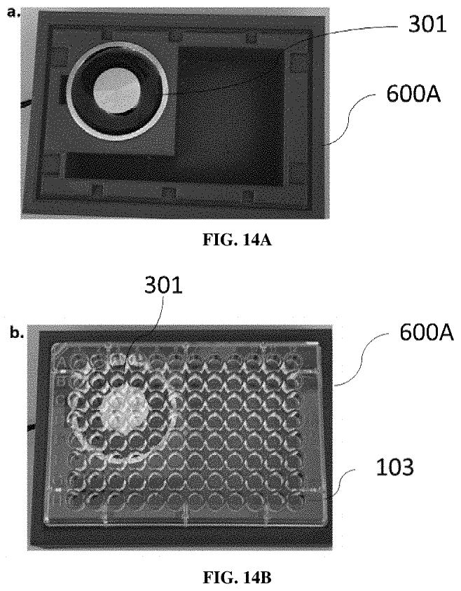

In some embodiments, the magnet (300) comprises an electromagnet (301) as shown in FIGS. 14A and 14B. In some embodiments, the electromagnet can generate an external magnetic field of at least 15 mT, (e.g., at least 20 mT, at least 25 mT, at least 30 mT, at least 35 mT, at least 40 mT, at least 45 mT, at least 50 mT, or at least 55 mT). In some embodiments, the electromagnet can generate an external magnetic field of 60 mT or less, (e.g., 55 mT or less, 50 mT or less, 45 mT or less, 40 mT or less, 35 mT or less, 30 mT or less, 25 mT or less, or 20 mT or less).

The electromagnet can generate an external magnetic field ranging from any of the minimum values described above to any of the maximum values described above. In some embodiments, the electromagnet can generate an external magnetic field of from 15 mT to 60 mT, (e.g, from 15 mT to 30 mT, from 15 mT to 40 mT, from 15 mT to 50 mT, from 30 mT to 40 mT, or from 40 mT to 60 mT).

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

Mechanical tissue environment is crucial in developing novel therapy concepts and tissue regenerative medicine. However, the understanding of tissue mechanical responsive mechanisms is substantially lacking, due to limited tissue models and resources available for well-controlled ex vivo study. Ferromagnetic soft composite materials offer controlled and untethered fast transformation of complex 3D shapes which is needed for such biomedical manipulation applications. Therefore, we introduce a smart, responsive 3D porous ferromagnetic interface that can reconstruct in vivo tissue microvibrational environment with desired full control of tissue dynamics. The objectives of these examples were to develop precise and controllable ferromagnetic porous composites; characterize the mechanical properties of smart responsive composites; distinguish the deformation capabilities of the responsive porous composites; verify the ability of the interface design to be suited for 3D cell culture with efficient waste and nutrient transport; establish the interface is compatible with 3D stem cell growth; and utilize 3D printing and particulate leaching techniques to develop high-fidelity microporous magnetic composite constructs with various micro patterns and shapes.

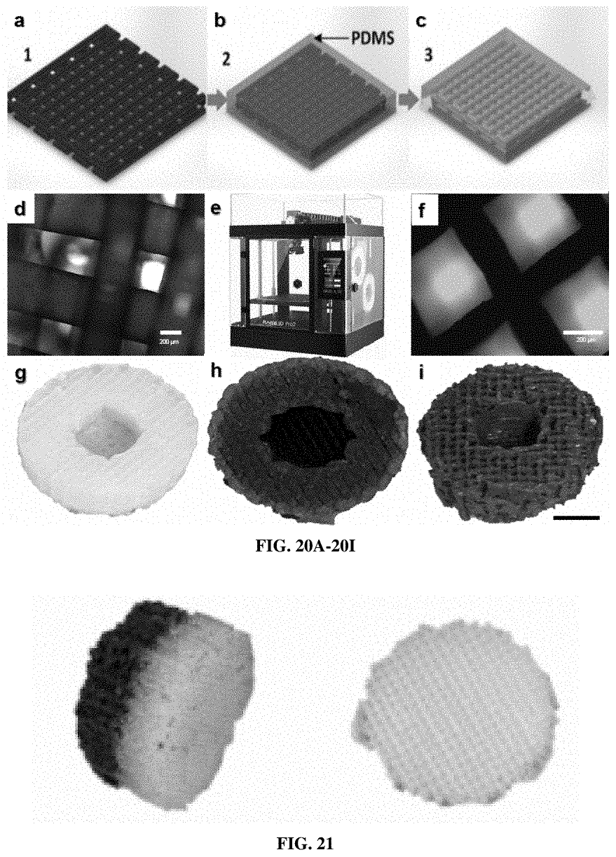

Cells respond to mechanical signals provided by the local extracellular matrix (ECM) and can control cell fate via cellular mechanotransduction. Therefore, it is important to be able to control and characterize the interface micromechanical environment. Utilizing 3D printing and particulate leaching techniques can be used to develop high-fidelity microporous magnetic composite constructs with various micro patterns and shapes (FIG. 20).

Ferromagnetic composites can be controlled via external magnetic fields and the correlation between magnetic field strength and deformation and pressure can be used to dictate the mechanical cues provided to a cellular network. The increased complexity allows for the generation of relevant dynamic stimuli that is experienced under biological conditions. This could be used to direct gene expression and cell fate through mechanosensitive cell receptors or channels.

### Example 1: Ferromagnetic Material with Remote Responsive Control for Dynamic Cell Culture

Described is a fabrication process that yields a well-ordered microporous magnetic elastomer composite that has controllable 4D transformation, or the ability to change the 3D conformation of the composites with time. The microporous magnetic elastomer has the capacity to induce tunable dynamic mechanical stimulation to a 3D cell culture.

Magnetic PDMS (Sylgard 184) composites are strong candidates for dynamic culture systems but are limited in their potential as a high-fidelity 3D platform given the highly viscous liquid precursor properties of PDMS that render these materials unsuitable for 3D printing. Therefore, we evaluated an indirect 3D printing fabrication technique to construct high-fidelity, remote-control microporous magnetic composites utilizing a 3D printed water-soluble sacrificial template for the fabrication for 3D PDMS tissue engineering scaffolds⁸. This process has not been utilized for the development of high-fidelity magnetic composites with low viscosity PDMS.

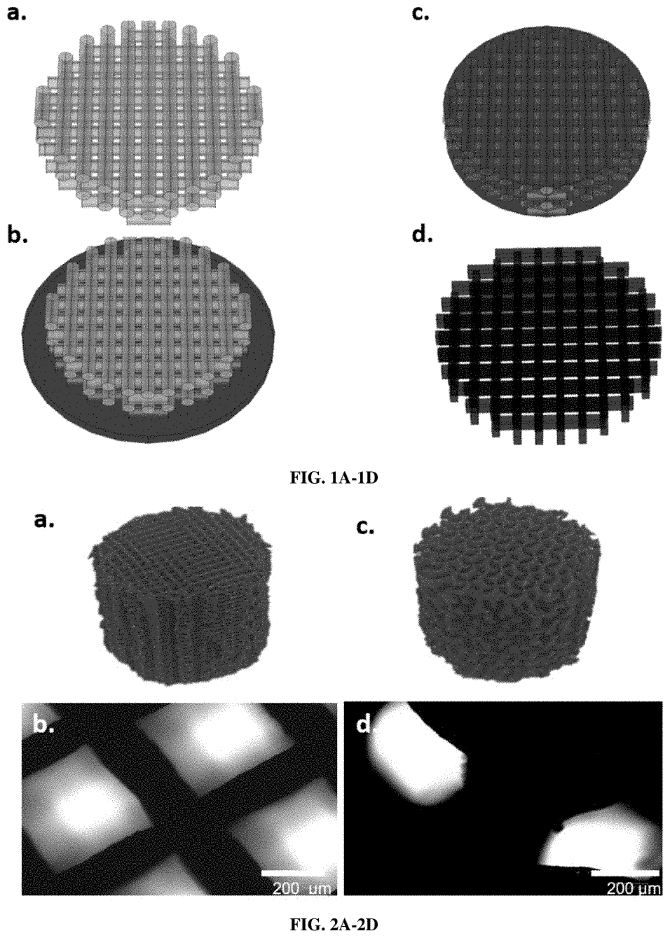

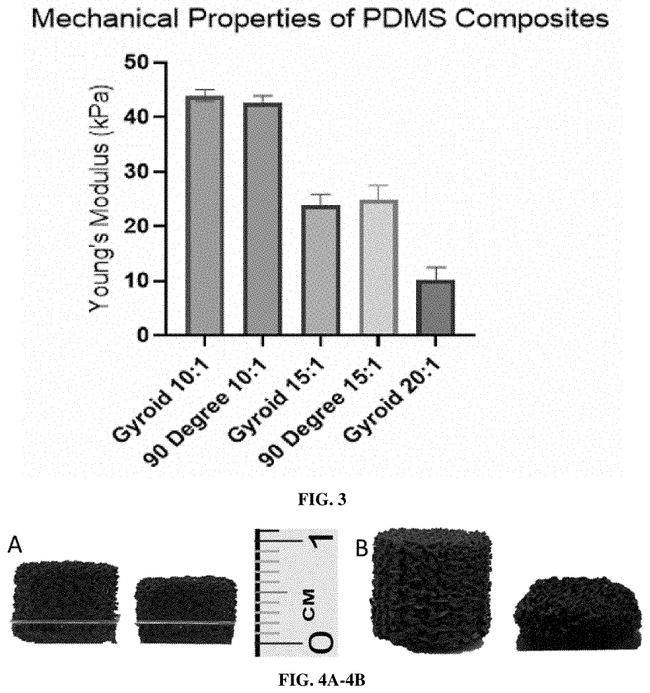

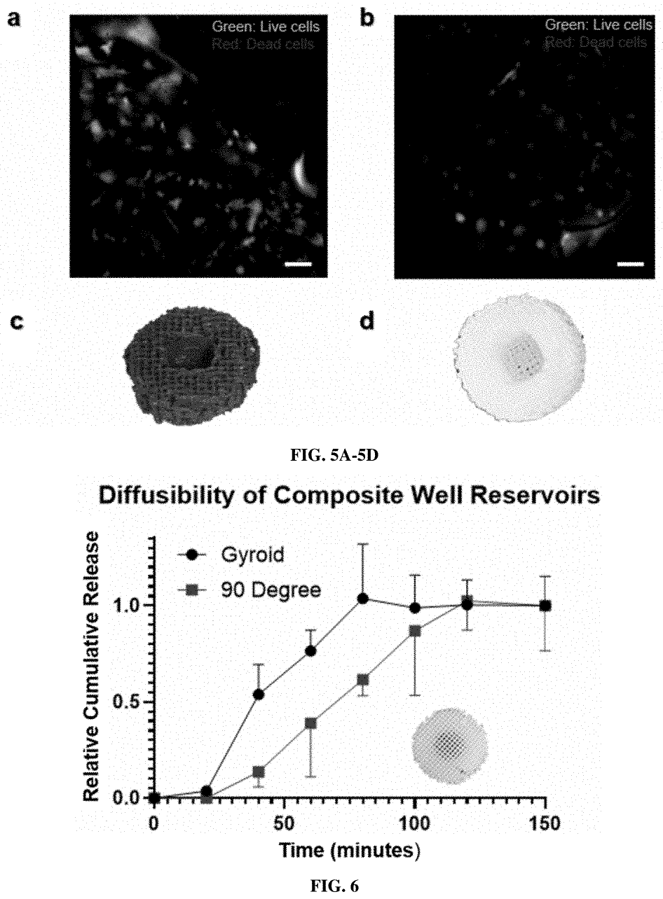

This fabrication technique allows for the assembly of magnetic porous constructs with controlled porosity and micropatterns, controllable dynamic mechanical manipulation, cell viability, and waste and nutrient transport for full interface culture conditions (FIG. 1, 2). Control of the micropatterns of the interface allows for fine control of adhesion events leading to an anisotropic dynamic topographic pattern actuation system. The patterning can influence not only cell fate programming, but spatial cell heterogeneity that is exhibited in certain tissue types. A controllable dynamic mechanical environment can be defined by the initial mechanical properties of the magnetic porous composite defined by porosity and magnetic particle density (FIG. 3). Then the dynamic mechanical stimuli can be controlled by the magnetic field strength, duration, and waveform and tunable via the strong actuation and fast transformation of the ferromagnetic composite (FIG. 4). The interface can provide a sustainable biocompatible substrate given the biocompatibility and bio inert characteristics of the PDMS leading to fixation of the acutely toxic magnetic microparticles fixed within elastomer solid (FIG. 5). If the porous composite was used as a reservoir type culture system or tissue engineering scaffold then the interconnected macropores will allow for cell infiltration, adhesion, and migration along with proper nutrient and waste supply given diffusion out of the porous magnetic composite and potential active mass transport via strain during activation of the composite (FIG. 6).

The flexibility in design allows for the integration of the magnetic composites into readily available cell culture platforms, such as well plates for high-throughput screening capabilities. Well plate compartmental devices can be designed to control different modes of magnetic actuation to the magnetic porous composites with the potential for both electromagnetic and permanent magnetically driven actuation. Electromagnetic actuation can lead to the development of a nanovibration environment that has been shown to have strong implications on the control of mesenchymal stem cell fate in both 2D and 3D cell cultures^{3,9,10}. Permanent magnet movable platforms can provide cyclic permanent magnetic actuation profiles that can lead to macroscopic deformation of the magnetic composites shown in FIG. 4. This can lead to the creation of both tensile and compressive strain cycles, depending on the sample used. Tensile and compressive strain has shown to be beneficial in programming the cellular behavior of mesenchymal stem cells in 3D cell cultures through the indication of differentiation markers for cartilage, bone, tendon, and muscle depending on the mechanical environment induced^{2,11-14}.

### DESCRIPTION OF THE CONSTRUCT

Construct design. In a first example, a construct including a magnetic composite is inserted at the bottom of 96-well plate. A 3D cell-laden gel can then be placed on top of the construct (see FIG. 18). This set-up allows for both strain and nano vibration actuations to be translated through the bottom surface of the cell culture. This can be a foundational actuation and can both provide for control and be simple to implement. It can also offer less variability in actuation given that it is constrained to the bottom of the well and has no distance variation in respect to the applied magnetic field.

In a second example, a non-magnetic composite (e.g., PDMS) can be inserted at the bottom of 96-well plate. The 3D cell-laden gel can then be placed on top of the non-magnetic composite. Then, a non-constrained magnetic composite can be placed above the cell laden gel (see FIG. 19). This set-up can allow for the greatest potential for strain given the “piston like” actuation of the magnetic composite and the tunable soft interfaces on the top and bottom of the 3D gel. This can add more variability given potential friction of the magnetic composite to the well surface and magnetic flux strength at different distance from the magnetic. These potential issues can be reduced by ensuring that the magnetic composite diameter is marginally less than the 96 well plate to ensure unrestricted actuation. The variation in magnetic field strength on the composite during actuation can be reduced by limiting the height of the 3D gel to ensure minimal distance variation between of the magnetic composite between on and off states.

The development of the smart responsive ferromagnetic porous composites for each sample is illustrated in FIG. 1. A fused filament fabrication printer (Pro2, Raised 3D) was used to print the PVA sacrificial templates. The 3D template structure was designed using a computer aided design (CAD) 3D slicer software provided by Raised 3D (ideaMaker). The 3D CAD design file then could be exported to the Pro2 by an .STL file to perform the 3D print. The method of printing the 3D templates include the PVA filament being fed through a heated extrusion nozzle that melts the thermoplastic and then solidifies on the heated stage bed after extrusion. The .STL computer slice file guides the nozzle to execute the print layer-by-layer by stacking xy planes on top of each other.

The printing infill percentage and infill patterns were varied to tune the mechanical properties, porosity, and topography of the interface design. The infill percentage refers to the amount of filament printed per layer with 100% infill being a filled structure with no pores. Given the PVA 3D print is a sacrificial template, the infill printed represents the pores of the final construct. A 70% infill percentage was chosen as this was the highest consistent attainable porosity for the final porous composite. The infill pattern indicates the micropattern printed to form the 3D geometric structure and both the rectangular grid with 90 degree offset every layer (FIG. 2A, 2B) and the gyroid patterns (FIG. 2C, 2D) were investigated.

The PVA printed template, shown in FIG. 1a., was then used to form the 3D magnetic porous composite structures. Carbonyl iron microparticles (Sigma-Aldrich) and PDMS (Sylgard 184, Dow Corning) were mixed in a 1:1 (w/w) ratio with varying base to cross-linking ratios of PDMS (10:1, 15:1, 20:1). The carbonyl iron/PDMS precursor solution was mixed thoroughly and degassed via centrifugation at 1000×g for 1 minute. The magnetic precursor was then poured in a well and the PVA template was placed on top of the magnetic precursor pool, shown in FIG. 1B. The PVA template and carbonyl iron/PDMS precursor were placed in a vacuum chamber at 400 kPa vacuum pressure for 2 hours to allow for the magnetic precursor to completely penetrate the pores of the PVA template via capillary action, the result is shown in FIG. 1C. The PVA templates with infiltrated carbonyl iron/PDMS precursor was then wiped of any excess magnetic precursor and then baked at 65° C. for 3 hours to allow for the carbonyl iron/PDMS precursor to cure within the template. The PVA template with infiltrated cured PDMS was then placed in a hot stir plate (above 70° C.) for 3-4 hours to allow for the PVA to completely dissolve. Once the PVA is completely dissolved remove the magnetic porous composites and allow time for them to completely dry. The final construct result is shown in FIG. 1D.

### DESCRIPTION OF THE ACTUATION DEVICE

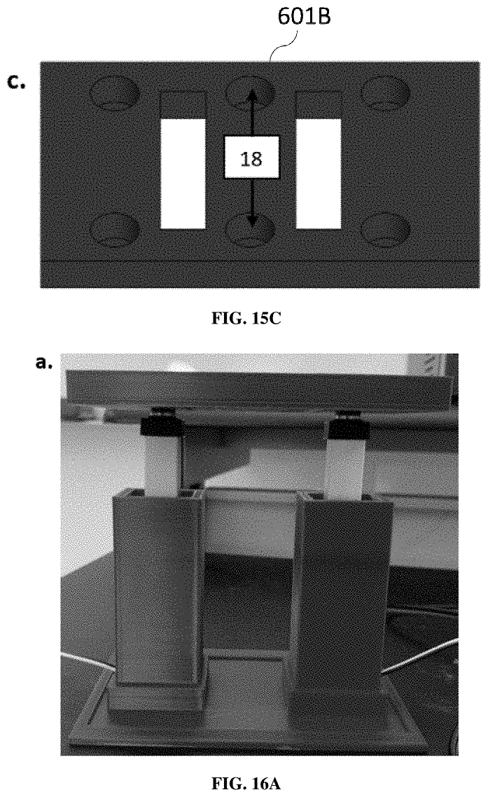

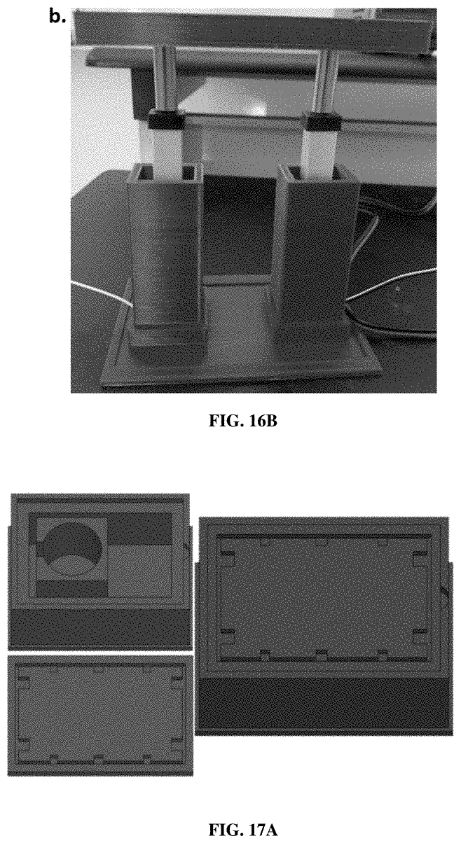

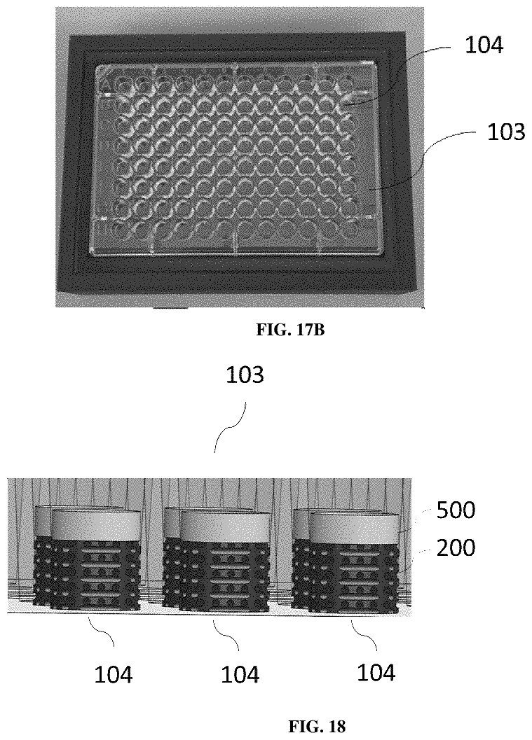

Actuation device designs. We developed two design iterations to encompass a 96-well plate with design flexibility for spatial control of a magnetic actuation. First iteration is a 3D Printed container that could enclose an electromagnet, microcontroller, and power supply with designed edges for the placement of a well plate (see FIG. 17A-17B). Second iteration is a 3D Printed container that would be open with a location for an electromagnet to be installed. A 96-well plate could be placed on the predesigned boundary indentations (see FIGS. 13 and 14A-14B).

The second iteration was used in the assays due to ease of implantation given the power supply and microprocessor being outside of the incubator; only requires two wires for power supply to the electromagnet; more control during the actuation process; and closer proximity of magnet to the 96-well plate given no lid compartment.

All magnetic constructs were designed to fit within a cell culture well plate. Well plate compartmental devices will be constructed to have control of the dynamic external magnetic environment placed on the magnetic constructs. The defined magnetic environment will control the strain level, frequency, and duration of actuation placed on the magnetic constructs that will be translated to the 3D cell culture. Two different modes of magnetic actuation will be investigated (nanovibration and strain) with the development of two different magnetic actuation devices.

Nanovibration will be the first mode of actuation investigated with the usage of electromagnets to generate external magnetic fields of 15-60 mT. These magnetic fields are unable to induce macrolevel actuation to the magnetic composites, however; they create micro and nanolevel vibrational environments due to the low external magnetic fields. The electromagnet could be modulated in a way to induce low frequency nano vibrations directly to a 3D stem cell culture. This is interesting for preconditioning 3D cell laden gels for potential tissue grafts. This has been studied by using piezo actuators to translate nanolevel deformations to the bottom of a well plate. The force developed by the magnetic composites will be assessed by studying the response of the materials at different electromagnetic fields with a low force sensor.

The nanovibration system can include a 3D printed housing (FIG. 13) that includes a compartment (e.g., circular compartment) to hold an electromagnet [13] that can be be level with the top surface of the housing. Contours and cuts are designed into the top of the housing [14] for a 96 well plate to be placed at a defined location with the surfaces of the 96 well plate. In some embodiments, the electromagnet and well plate surfaces are in contact ensuring the ability to induce the highest range of magnetic actuation. An aperture [15] was then designed to allow for the external wires of the electromagnet to be supplied out of the device. A power supply, relay, and microcontroller, outside the incubator, can be used to control the magnetic field strength, frequency, and duration. This system was used for all the samples except Sample 2 given the distance constraints with magnetic fields. The final construction is displayed in FIG. 14 without (FIG. 14A) and with (FIG. 14B) a 96 well plate.

A permanent magnet system to induce macrolevel constraints can include a micro linear actuator driven stage that can move permanent magnets in the vertical direction. The movement of the stage can create a dynamic magnetic field profile that can induce macrolevel strain, as shown in FIG. 4, on the magnetic porous constructs due to the larger external magnetic field. This system can induce many different modes of strain depending on the sample used. Samples 1 and 2 can induce uniaxial tensile (Sample 1) and compressive (Samples 2 & 3) profiles. Samples 4-5 can induce complex mechanical stimuli on the 3D cell culture; computational modeling can be used to identify the complete mechanical environment induced on the cell culture.

The 3D printed device for the permanent magnet system can include a housing that has two inner pillars [16] that are designed to fit the curvature of two micro linear actuators (FIG. 15). Apertures are developed in both the pillars and the outer walls of the housing for the wires to be directed out of the housing. Contours and cuts are made at the very top of the compartment to hold a 96 well plate at a defined location with the surfaces of the wells at the same level as the top surface of the container [14]. A stage was printed to have spaces on the bottom surface to fit the stroke ends of each of the linear actuators [17]. Circular wells are designed on the top to hold permanent magnets in spatial controlled locations [18]. The height of the housing can vary depending on vertical translation potential of the stage that is dependent on the linear actuator stroke length. A power supply, multi-channel relay switch, and microcontroller can be used to control the states of each relay switch channel to control the upward and downward movement of the linear actuators which directs the position of the stage. The frequency, amplitude, and duration of the dynamic movement of the stage will be defined by the parameters set for the microcontroller.

By way of example, for the induction of strain, 200-400 mT applied magnetic fields can be used to deform the magnetic material (e.g., an elastomer such as PDMS containing NdFeB microparticles dispersed therein). Permanent magnets can be used to create a pulsated magnetic field via moving the stage up and down. The linear actuators can perform a 1 Hz pulsated field with 7-8 mm translation (speed of linear actuators=14-15 mm/s).

### Advantageous Effects

In the case of Samples 1 and 2, a porous PDMS/magnetic composite and PDMS porous construct can be interfaced at the top and bottom of a 3D cell culture. An external magnetic field can actuate the PDMS/magnetic composite to allow for control of the pressure placed on a 3D cell culture. Variability in composition of the magnetic composite can allow for adjustment of the initial mechanical properties of the soft composites and force potential to mimic in vivo specific mechanical environments as shown by FIG. 3. The well-defined porous interface can allow for anisotropic mechanical stimuli via micro/nanopatterns on the interface to have both spatial and temporal control of cell behavior.

In the case of Sample 3, a PDMS/magnetic composite could provide an interface system that could be in full contact of a 3D cell culture. The cell culture could be inserted into a magnetic/PDMS porous reservoir and an external magnetic field could be used to control the mechanical stimuli placed on the cell culture with defined micropatterns. This system is viable due to the interconnected porosity of magnetic reservoir system allowing for waste and nutrient transport (FIG. 6.) that shows the diffusional mass transport from the reservoir to the bulk fluid.

Sample 4 includes a 3D magnetic porous composite scaffold that can be seeded with cells. Then the magnetic composite interface could directly transmit mechanical stimuli to the adherent cells and computational modeling and experimental techniques to develop well controlled and defined micromechanical environments and study cellular behavior. The development of these models could be obtained by utilizing the works described by Mokhtari-Jafari, F. et al., Zhao, R., et al., and Malvè, M., Bergstrom, D. J. & Chen, X. B. ¹⁵⁻¹⁷ This can allow for the study of the structural and fluid mechanics at the microscale level within the scaffold to best simulate the forces being applied at the structural and fluid mechanic interface. To our knowledge, this has never been studied with a bioinert actuating scaffold.

Sample 1

Sample 1 includes a 3-D PDMS magnetic porous composite that is fabricated to establish a dynamic interface for a 3D cell culture. The magnetic composite is designed to be inserted into the bottom of well plate and provide a surface contact for a 3D cell culture gel that can placed on top. Modifications can be made to the surface of the magnetic composite to increase absorption of the gel to the top surface of the magnetic composite. This can permit the most ideal dynamic interaction translation between the magnetic composite and the gel. A microcontroller is used to control the magnetic field strength, waveform, frequency, and duration. The external magnetic field created will influence the dynamic tensile strain or vibration environment placed on the 3D cell culture.

Sample 2

Sample 2 includes a non-magnetic (PDMS) porous construct and a 3-D PDMS magnetic porous composite (FIGS. 7A and 7B) fabricated to create a dynamic interface dual contact system for a 3D cell culture. The 3D cell culture gel [3] can be contacted on the top by the magnetic porous composite [2] and the PDMS only porous construct on the bottom [1], and these can be designed to fit a well plate insert [4]. The culture inserts allow for waste and nutrient exchange in the radial and azmithual directions. An external magnetic field is applied to tune the dynamic mechanics and pressure of the magnetic composite on the top surface of the cell culture which is fixed by the PDMS only construct below. The compressive force induced on the 3D cell culture gel is depicted in FIG. 8, as an external magnetic field induces the magnetic composite to deform and prompts a pressure on the 3D gel.

Sample 3

Sample 3 has the same set-up as the Sample 2 but the surfaces of both porous constructs are functionalized. The interface of the constructs includes a full infill or nanopatterned thin membrane that can be developed using photolithography (FIG. 9). This can enable the tunability to provide specific temporal stimulation and influence cellular alignment.

Sample 4

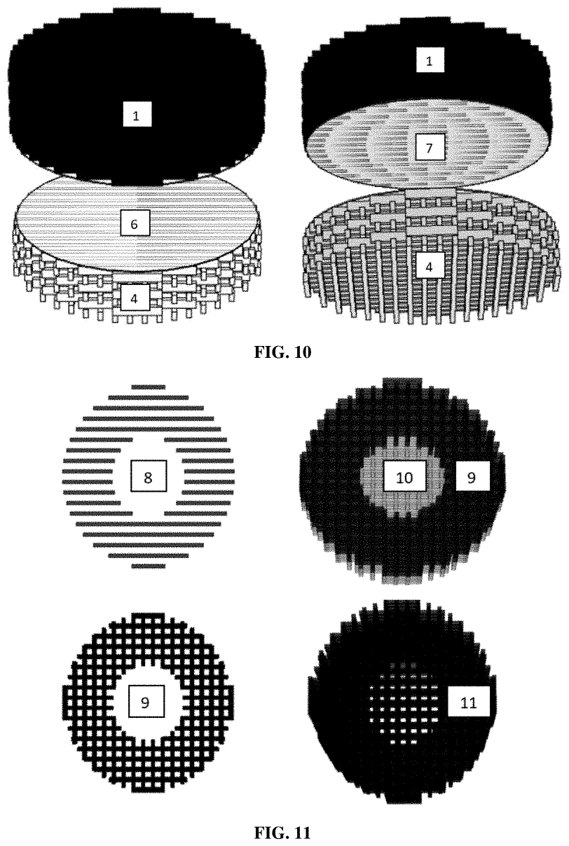

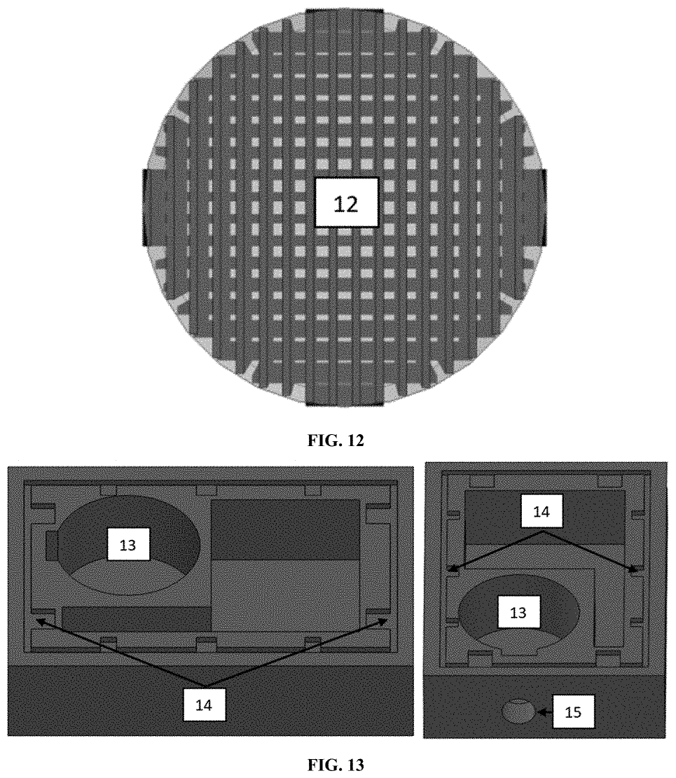

Sample 4 includes a magnetic composite reservoir construct that can provide 360 degrees 3D dynamic actuation by being in full contact with a cell culture. The 360 degrees actuation system is described in FIG. 10. and a cell culture is inserted via an injectable hydrogel. A finite element model can be used to study and regulate the mechanical environment experienced by the interfaced cell culture.

Sample 5

Sample 4 includes a dynamic bioinert magnetic porous scaffold system that can provide a programmable microenvironment to cells. The cells are seeded on the scaffold material and the microporous pores allow for the cells to migrate throughout the 3D construct (FIG. 11). Magnetic actuated mechanical stimuli then can be transferred to the cells to study cell behavior to specific 3D mechanical microenvironments.

Additionally, FIG. 21 shows a simple interface system to have full control of the interface dynamics of the composite by contacting the top and bottom of a 3D cell matrix. The ability of the composite interface material to induce perfusion via mechanical stimuli can be observed. Different mechanical stimuli produced by the interface system can be studied for how it can control stem cell fate within a 3D cell culture.

Magnetic Field Modeling

Magnetic Flux Density Modeling can be used to calculate the magnetic flux density at certain locations from a magnet. This can help with size constraints of the porous PDMS and magnetic composite constructs along with the height of the 3D gel. Increased or varying location of the magnetic composite away from the magnetic will change the magnetic field applied and the sequential response. These models can help to ensure that the proper magnetic field is applied given the location of the magnetic composites in respect to the magnet.

Force Response

Assessment of Force Translation of Magnetic Composites. A 0-11b small force sensor can be used to detect the amount of force the magnetic composites generate at different flux densities. The force readings can be measured via both an electromagnet and permanent magnets at different distances from the magnetic composite.

A Magnetic Flux Modeling and a Gauss Meter can be used to access the magnetic fields at different distances. This is needed to access the nano vibration actuation potential and the driving force of the piston-like set-up for the magnetic composites.

### REFERENCES

1 Engler, A. J., Sen, S., Sweeney, H. L. & Discher, D. E. Matrix Elasticity Directs Stem Cell Lineage Specification. Cell 126, 677-689, doi:10.1016/j.cell.2006.06.044 (2006).
2 Liu, H. et al. Microdevice arrays with strain sensors for 3D mechanical stimulation and monitoring of engineered tissues. Biomaterials 172, 30-40, doi:10.1016/j.biomaterials.2018.04.041 (2018).
3 Campsie, P. et al. Design, construction and characterisation of a novel nanovibrational bioreactor and cultureware for osteogenesis. Scientific Reports 9, doi:10.1038/s41598-019-49422-4 (2019).
4 Occhetta, P. et al. Hyperphysiological compression of articular cartilage induces an osteoarthritic phenotype in a cartilage-on-a-chip model. Nature Biomedical Engineering 3, 545-557, doi:10.1038/s41551-019-0406-3 (2019).
5 Lui, Y. S. et al. 4D printing and stimuli-responsive materials in biomedical aspects. Acta Biomaterialia 92, 19-36, doi:10.1016/j.actbio.2019.05.005 (2019).
6 Jackson, J. A. et al. Field responsive mechanical metamaterials. Science Advances 4, doi:10.1126/sciadv.aau6419 (2018).
7 Kim, Y., Yuk, H., Zhao, R., Chester, S. A. & Zhao, X. Printing ferromagnetic domains for untethered fast-transforming soft materials. Nature 558, 274-279, doi:10.1038/s41586-018-0185-0 (2018).
8 Mohanty, S. et al. Fabrication of scalable and structured tissue engineering scaffolds using water dissolvable sacrificial 3D printed moulds. Materials Science and Engineering: C 55, 569-578, doi:10.1016/j.msec.2015.06.002 (2015).
9 Marycz, K. et al. Low-frequency, low-magnitude vibrations (LFLM) enhances chondrogenic differentiation potential of human adipose derived mesenchymal stromal stem cells (hASCs). PeerJ 4, doi:10.7717/peerj.1637 (2016).
10 McClarren, B. & Olabisi, R. Strain and Vibration in Mesenchymal Stem Cells. Int J Biomater 2018, 8686794, doi:10.1155/2018/8686794 (2018).
11 Charoenpanich, A. et al. Cyclic Tensile Strain Enhances Osteogenesis and Angiogenesis in Mesenchymal Stem Cells from Osteoporotic Donors. Tissue Engineering Part A 20, 67-78, doi:10.1089/ten.tea.2013.0006 (2014).
12 Guo, T. et al. Effect of Dynamic Culture and Periodic Compression on Human Mesenchymal Stem Cell Proliferation and Chondrogenesis. Annals of Biomedical Engineering 44, 2103-2113, doi:10.1007/s10439-015-1510-5 (2015).
13 Choi, J. R., Yong, K. W. & Choi, J. Y. Effects of mechanical loading on human mesenchymal stem cells for cartilage tissue engineering. Journal of Cellular Physiology 233, 1913-1928, doi:10.1002/jcp.26018 (2018).
14 Nam, H. Y., Pingguan-Murphy, B., Abbas, A. A., Merican, A. M. & Kamarul, T. Uniaxial Cyclic Tensile Stretching at 8% Strain Exclusively Promotes Tenogenic Differentiation of Human Bone Marrow-Derived Mesenchymal Stromal Cells. Stem Cells International 2019, 1-16, doi:10.1155/2019/9723025 (2019).
15 Mokhtari-Jafari, F. et al. Mathematical modeling of cell growth in a 3D scaffold and validation of static and dynamic cultures. Engineering in Life Sciences 16, 290-298, doi:10.1002/elsc.201500047 (2016).
16 Zhao, R., Kim, Y., Chester, S. A., Sharma, P. & Zhao, X. Mechanics of hard-magnetic soft materials. Journal of the Mechanics and Physics of Solids 124, 244-263, doi:10.1016/j.jmps.2018.10.008 (2019).
17 Malvè, M., Bergstrom, D. J. & Chen, X. B. Modeling the flow and mass transport in a mechanically stimulated parametric porous scaffold under fluid-structure interaction approach. International Communications in Heat and Mass Transfer 96, 53-60, 2018.05.014 (2018).

### Example 2: Remote-Controlled 3D Porous Ferromagnetic Interface Towards High-Throughput Dynamic 3D Cell Culture

Abstract

Mechanical stimuli have been shown to play a large role in cellular behavior, including cellular growth, differentiation, morphology, homeostasis, and disease. Therefore, developing bioreactor systems that can create complex mechanical environments for both tissue engineering and disease modeling drug screening is appealing. However, many of existing systems are restricted due to their bulky size with external force generators, destructive microenvironment control, and low throughput. These shortcomings have preceded to the utilization of magnetic stimuli responsive materials, given their untethered, fast, and tunable actuation potential at both the microscale and macroscale level, for seamless integration into cell culture wells and microfluidic systems. Nevertheless, magnetic soft materials for cell culture have been limited due to the inability to develop well-defined 3D structures for more complex and physiological relevant mechanical actuation. Herein, we introduce a facile fabrication process to develop magnetic-PDMS (polydimethylsiloxane) porous composite designs with both well-defined and controllable microlevel and macrolevel features to dynamically manipulate 3D cell-laden gel at the scale. The intrinsic stiffness of the magnetic-PDMS porous composites is also modulated to control the deformation potential to mimic physiological relevant strain levels, with 2.89 to 11% observed in magnetic actuation studies. High cell viability was achieved with the culturing of both human adipose stem cells (hADMSCs) and human umbilical cord mesenchymal stem cells (hUCMSCs) in 3D cell-laden gel interfaced with the magnetic-PDMS porous composite. Also, the highly interconnected porous network of the magnetic-PDMS composites facilitated free diffusion throughout the porous structure showcasing the potential of a multi-surface contact 3D porous magnetic structure in both reservoir and 96-well plate insert designs for more complex dynamic mechanical actuation. In conclusion, these studies provide a means for establishing a biocompatible, tunable magnetic-PDMS porous composite with fast and programmable dynamic

Introduction

Cells have been shown, especially mesenchymal stem cells (MSCs), to both sense and act upon their mechanical environment via a mechanism called mechanotransduction¹⁻⁷. This process consists of the cell being able to convert mechanical cues into intracellular biochemical signals for driving cellular growth, differentiation, morphology, homeostasis, and disease. Mechanical stimuli experienced by cells can come in both intrinsic and extrinsic forms. Current studies have looked at primarily one dependent force variable at a time to understand how each stimuli affects cellular behavior¹⁻². Most of these investigations utilized extrinsic forces to manipulate the intrinsic behavior of the cells through actomyosin contractility and cytoskeleton rearrangement, which can trigger biochemical signals depending on the dynamic nature of the force³⁻⁵. There are many different platforms to mimic in vivo physiological relevant forces experienced on cell cultures including substrate stiffness⁶⁻⁷, micro/nanopattering⁸⁻¹⁰, hydrostatic pressure¹¹⁻¹², perfusion flow¹³⁻¹⁴, shear forces¹⁵⁻¹⁶, compressive forces¹⁷⁻²⁰, tensile forces²¹⁻²³, and vibration²⁴⁻²⁶. The findings have led to the development of bioreactor systems for providing specific and complex mechanical environment used in tissue engineering and disease modeling²⁷⁻²⁸.Some developed microscale systems can overcome the complications involved with conventional bioreactor systems which are bulky, lack microenvironment control, and have low experimental throughput^{19, 25, 30-31} The mechanical stimuli to 3D cell cultures also can be achieved via pneumatic actuation of a PDMS membranes^{19,31}, dielectric elastomer actuators³²⁻³³, and magnetic elastomer actuation³⁴⁻³⁷ in high-throughput microfluidic, micro array, or cell culture well-plate systems. However, the current existing microscale systems to date are still lacking the capacity for micro level spatial control of mechanical actuation with most needing external forces to act upon an interface substrate.

Microscale actuator systems are critically needed in evaluating and developing physiological relevant actuation to cell cultures with multiple modes and ranges of actuation. Herein, we introduced a magnetic stimuli responsive materials to bridge such technology gap. Although magnetic materials have shown to provide highly tunable, predictive, complex transformation potential at both the macroscopic and microscopic level³⁸⁻⁴⁰, developing magnetic elastomer actuators with defined 3D structures and porosity using PDMS (polydimethylsiloxane) for dynamic cell culture is emerging^{6-10, 19, 34-36, 41}. Investigators have explored the development of magnetic porous composites as devices for on-demand drug delivery by the usage of sacrificial molds of salt or sugar microparticles to define the porous network⁴²⁻⁴³. Yet, there has been minimal investigation into porous magnetic composites as scaffold or porous interfaces with high-fidelity porous networks for cell culture considerations. Therefore, we developed a high-fidelity, tunable magnetic PDMS porous composite that can be actuated remotely via an external magnetic field. To our knowledge, this will be the first magnetic PDMS porous composite with well-defined 3D microstructures for full control over the porosity and micropatterns to study the topographical cues in 3D cell culture. Because of the ability to precisely manipulate the intrinsic stiffness and magnetic particle density in the porous magnetic PDMS composites, the resulted physiological relevant strain level enables the potential control of human umbilical cord mesenchymal stem cells (hUCMSCs) differentiation. Also, the porous magnetic PDMS showed to be a suitable 3D cell culture substrate with high cell viability for both adipose-derived mesenchymal stem cells (hADMSCs) and hUCMSCs in a 3D fibrin gel. The highly interconnected porous network of the magnetic PDMS porous composites enhances free diffusion through the material unlocking the potential to use as the multiple surface contact porous constructs for more complex dynamic mechanical actuation. With this platform using remotely controllable porous materials, small and complex cell culture magnetic interfaces can be implemented into standard cell culture 96 well plate as a potential high-throughput platform for advancing dynamic 3D cell culture systems.

Materials and Methods

FDM 3D Printing of Sacrificial Templates

A fused filament fabrication printer (N2, Raise 3D), or fused deposition modeling (FDM), was used to print the PVA sacrificial templates. The 3D template structure was designed using a computer aided design (CAD) 3D slicer software provided by Raise 3D (ideaMaker). The 3D CAD design file then could be exported to the printer by a .STL file to perform the 3D print. The method of printing the 3D templates include the PVA filament being fed through a heated extrusion nozzle that melts the thermoplastic and then solidifies on the heated stage bed after extrusion. The .STL computer slice file guides the nozzle to execute the print layer-by-layer by stacking x-y planes on top of each other. The printing infill percentage and infill patterns were varied to tune the mechanical properties, porosity, and topography of the interface design. The infill percentage refers to the amount of filament printed per layer with 100% infill being a filled structure with no pores. Given the PVA 3D print is a sacrificial template, the infill printed represents the pores of the final construct. A 70% infill percentage was chosen as this was the highest consistent attainable porosity for the final porous composite. The infill pattern indicates the micropattern printed to form the 3D geometric structure and both the rectangular grid and gyroid patterns were investigated. The printing parameters are shown in Table 1.

TABLE 1
PVA FDM 3D Printing Parameters for the
fabrication of porous PVA templates.
PVA Printing Parameters

| Nozzle Diameter | 400 | μm |
|---|---|---|
| Infill Pattern | Grid and Gyroid | |
| Layer Height | 150 | μm |
| Infill Density | 30, 50, 70% | |
| Extruder Temperature | 190° | C. |
| Bed Temperature | 60° | C. |

3D Fabrication of Magnetic Porous Composites

The PVA printed templates were used to form the 3D magnetic porous composite structures. Carbonyl iron microparticles (Sigma-Aldrich) and PDMS (Sylgard 184, Dow Corning) were mixed in a 1:1 (w/w) ratio with varying base to curing agent ratios of PDMS (10:1, 15:1, 20:1). The carbonyl iron/PDMS precursor solution was mixed thoroughly and degassed via centrifugation at 1000×g for 1 minute. The physics of magnetic actuation and properties were characterized previously. The magnetic precursor was then poured in a well and the PVA template was placed on top of the magnetic precursor pool. The PVA template and carbonyl iron/PDMS precursor were placed in a vacuum chamber at 400 kPa vacuum pressure for 2 hours to allow for the magnetic precursor to completely penetrate the pores of the PVA template via capillary action. The PVA templates, with infiltrated carbonyl iron/PDMS precursor, were then wiped of any excess magnetic precursor and then baked at 65° C. for 4 hours to allow for the carbonyl iron/PDMS precursor to cure within the template. The PVA template with infiltrated cured PDMS was then placed in a hot stir plate (above 70° C.) for 3-4 hours to allow for the PVA to completely dissolve. Once the PVA is completely dissolved, the magnetic-PDMS porous composites could be removed and left out to dry.

Mechanical Analysis of Magnetic Porous Composites

Mechanical analysis of the magnetic-PDMS porous composites was tested by performing controlled axial compressions utilizing an ElectroForce 5500 (TA Instruments). The testing parameters including a controlled axial compression at 0.01 mm/s of cylinder composites of the gyroid infill pattern (8.5 mm×7.8 mm) and grid infill pattern with 90-degree layer offset (9 mm×7.8 mm). The Young's modulus of the composites were determined by the linear portion of the stress vs strain curve which was determined to be 2% to 10% strain.

Controlled Deformation Characterization of Magnetic Composites Controlled deformation studies were performed to assess the ability to induce strain on the magnetic-PDMS composites with varying magnetic fields. A 3D printed compartmental box was printed to hold both a phone and 96-well plate at a defined distance and location to have reproducible image collection for both qualitative and quantitative strain analysis. Grid (6.8×5 mm) and gyroid (7.2×5 mm) PVA templates were prepared to make magnetic composite structures to fit inside a 96-well plate. 15:1 (PDMS base: cure) grid and 20:1 (PDMS base: cure) gyroid magnetic composites were fabricated and used for controlled deformation studies. The 3D printed box was designed to have a permanent magnet insert location. 3D printed blocks were placed underneath the permanent magnet to adjust the applied magnetic field to the magnetic composites within the 96 well plate. Applied magnetic fields of 0, 240, 325, and 415 mT were performed on the magnetic-PDMS composites in a 96-well plate with DPBS media. Image collection was obtained via a 12-megapixel f/1.8 camera iPhone XR, Apple). Applied magnetic fields were measured via a gaussmeter (TD8620 Handheld Digital Gauss TeslaMeter, China),

Dynamic actuation studies of the magnetic composites were performed using a 3D printed box that was designed to develop a dynamic magnetic field profile to magnetic composites within a 96-well plate. The box was built to hold two micro linear actuators that drive a stage, encompassing permanent magnets, in the z-direction to control the frequency, amplitude, and duration of an applied magnetic field experienced by the magnetic porous composites. A cyclic, 325 mT magnetic field was used for all the magnetic actuation studies. Magnetic porous composite recovery studies were performed by imaging the height of both gyroid (20:1) and grid (15:1) under no applied magnetic field after different duration of dynamic magnetic field actuation cycles (0, 5, 25, 50, 100). On-demand precise actuation analysis of the magnetic porous composites was executed by inducing a pause function at the maximum applied magnetic field during the dynamic magnetic actuation cycle for imaging the strain levels of the gyroid (20:1) and grid (15:1) for the same actuation cycle durations studied for the recovery tests.

Cell Culture of hUCMSCs and hADMSCs

Patient derived human umbilical cord stem cells (hUCMSCs) and adipose stem cells (hADMSCs) were provided by the Midwest Stem Cell Therapy Center (MSCTC). Cells were maintained in Gibco™ DMEM, high glucose, pyruvate media supplemented with 15% fetal bovine serum and 1% penicillin-streptomycin, all purchased from Gibco™, Dublin, Ireland. Cells were cultured in a humidified incubator at 37° C. and 5% CO₂. Cells were detached by the incorporation of trypsin-EDTA (Gibco™) followed by the addition of cell culture media, and then centrifuged for 5 minutes at 1500 rpm. Passages 3-5 of hUCMSCs were used in 3D fibrin gel preparation.

Fibrin hUCMSC Laden 3D Gel Preparation

Stock solution aliquots of fibrinogen (Millipore Sigma, US) and thrombin at concentrations of 45 mg/mL and 10 U/mL were used in the preparation of the fibrin gels. The aliquots were heated up to 37° C. prior to preparation. The fibrinogen stock solution is then diluted with DPBS and mixed gently to give a final concentration of 22.5 mg/mL of fibrinogen. DPBS is also added to the thrombin stock solution to yield a final concentration of 5 U/mL thrombin solution. The pelleted cells were then suspended and mixed carefully into the thrombin solution. 50 μL of the fibrinogen preparation is then added to the top of the porous constructs followed by the addition of the 50 μL of the thrombin-cell suspension. The gel is then mixed with the pipette tip immediately given the rapid polymerization and placed in the cell incubator for 15 minutes to ensure full polymerization. Cell culture media is then added on top of the gel and placed back in the incubator. The final fibrinogen and thrombin concentrations of the gels are 11.25 mg/mL and 2.5 U/mL respectively with 12,000 cells per gel preparation.

3D Cell Culture for Cell Viability Testing

Grid (3×6.8 mm) and gyroid (3×7.2 mm) cylinder PVA structures were printed to fit at the bottom of a 96-well plate well. Porous composites were fabricated as previously described with 8 different groups for cell viability analysis. The groups were represented by the two different infill patterns (grid, gyroid), two PDMS crosslinking ratios (10:1, 15:1), and both magnetic and PDMS porous constructs. For cell viability studies, the porous magnetic and PDMS constructs were sterilized by a 70% ethanol bath followed by UV exposure. The constructs were thoroughly washed with DPBS before insertion into a cell culture plate well.

Fibrin hADMSC laden 3D Gels were prepared as described for the hUCMSC laden 3D gel, while using hADMSCs, with a fibrinogen and thrombin concentrations of 10 mg/mL and 2.5 U/mL. Fibrin gels were cultured in both magnetic and PDMS only 3D porous well constructs. The 3D porous well design consisted of a 4 mm diameter and 2 mm tall well formed in a cylinder with 3 mm thick walls. The fibrin gels were cultured for 5 days in 48 well tissue culture plates.

For qualitative and quantitative analysis of the cell viability of the different porous construct groups and the no construct control group, a live/dead-assay was performed using a live/dead imaging kit (LIVE/DEAD® Viability/Cytotoxicity Kit, Invitrogen™). The live/dead staining was performed on the cell cultures on the fourth day under cell culture conditions. The attached hUCMSC cell-laden fibrin gels attached to the porous constructs were removed from the 96 well-plate and flipped over and placed at the bottom of the wells for imaging. Given the high porosity and thin nature of the constructs, they provided sufficient transparency for imaging without removing the construct from the gel. As for the magnetic well cell culture studies, hADMSC cell-laden fibrin gels were removed from the well constructs with a spatula and placed in the bottom a 96-well plate for imaging.

For staining, 20 μL of 2 mM EthD-1 and 5 μL of 4 mM calcein AM from the LIVE/DEAD® reagents were added to 10 mL of DPBS. The resulting working dye was then vortexed to ensure thorough mixing and a final concentration of 4 μM EthD-1 and 2 μM calcein AM was achieved. Cell culture media was then aspirated from the well of each sample and 100 μL of the live-dead solution was added to each well. The well-plate was then covered with aluminum foil and incubated for 25 minutes. After incubation, the live/dead solution was aspirated from each well and each sample was washed with DPBS and imaged with a fluorescent microscope (EVOS, Invitrogen and Primovert, ZEISS). Qualitative and quantitative analysis was performed by using post-processing imaging software (Fiji, ImageJ).

Diffusivity Studies

To determine the viability of the ferromagnetic interface system to be used as a 360-degree interface system, the ability for waste and nutrients to diffuse through the interconnected micropores of the magnetic porous required investigation. Therefore, a diffusivity study of the composite interfaces was performed by developing a magnetic composite reservoir with a 5×5 mm cylinder well with 3 mm thick porous walls. The diffusivity would be measured by the absorbance of a 5% (w/w) alginate sol gel (Alginic acid, Fischer Scientific) with 15 mg/mL of aniline blue (Fischer Scientific) in the bulk fluid. The reservoirs were first submerged in 1 mL of PBS in 24 well plates and preconditioned via physical agitation to allow for PBS to enter the pores of magnetic porous composites. The alginate/aniline blue sol gel was then loaded in a 1 mL syringe with a 26 G needle and 100 μL of solution was injected into each magnetic composite reservoir, followed by the addition of another 800 μL of DPBS. The cumulative mass transport out of the magnetic reservoir composites was measured by removing 200 μL for absorbance analysis of the well plate bulk solution outside the composite interface at specified time points (20, 40, 60, 80, 100, 120, and 150 min), 200 μL of PBS was added after each time point. Studies were completed in triplicates for both grid and gyroid reservoirs magnetic composites.

Cumulative Release Profile

A calibration curve was found by performing seven 1:1 serial dilutions of 1.75 g/mL of aniline blue and then measuring the absorbance at 625 nm of 200 uL of each dilution with a plate reader (Synergy H1 Hybrid Multi-Mode Reader, BioTek). Absorbance levels for PBS were also measured to allow for the calculation of relative absorbance values from the diffusivity studies. Release profiles were found by measuring the absorbance at 625 nm of the aliquots taken at each time point with a plate reader. Relative increase in the absorbance profiles for each time point were then calculated by subtracting the measured value by the previous absorbance level accounting for the addition of 200 μL after each removed sample. Cumulative release profiles were then established by calculating the cumulative sum of the relative increase values.

Results

Facile and Versatile Fabrication of 3D Porous Ferromagnetic Interface

Combining 3D printing and particulate leaching methods, we were able to fabricate high-fidelity 3D porous magnetic composites. We used PDMS to develop a soft porous magnetic substrate actuator for micromechanical actuation of 3D dynamic cell cultures⁴⁴. Both grid and gyroid infill patterns were investigated for precise control and high-fidelity fabrication of 3D magnetic-PDMS porous constructs. The grid pattern structure was chosen given it is a widely used pattern in many different extrusion based tissue engineering scaffolds. As for substrate interface considerations, rectangular grid patterns have been widely studied to induce anisotropic cell morphology in both 2D static¹⁰ and time dependent 3D cell culture surfaces⁴⁵. The grid structure is widely used given there are many different tissue types in the mesoderm lineage that have anisotropic cellular network formations that align in a linear fashion. The gyroid infill pattern was also investigated as triply minimal surfaces have emerged as a 3D printed pattern for porous biomaterial fabrication. Gyorid structures have been shown to have good mechanical properties, and due to their minimal surface, they allow for high permeability and nutrient and waste transport⁴⁶⁻⁴⁷. The facile fabrication with simple three steps were illustrated in FIG. 28A, which can produce versatile, precisely controlled microstructure patterns. The resulting 3D constructs were shown in FIG. 22D-22F, which exhibited very uniform and regular 3D microstructure pores. The entire fabrication is straightforward, and only takes about twelve hours to complete with high throughput capabilities as many 3D magnetic composites can be developed at the same time. Most importantly, the microscale porous structures can be precisely control via a 3D FDM printer with feature sizes under 200 μm, 1 μm x-y axis precision, and layer resolution around 100 μm (FIGS. 22G and 22I) which is well suited for dynamic cell culture actuation.

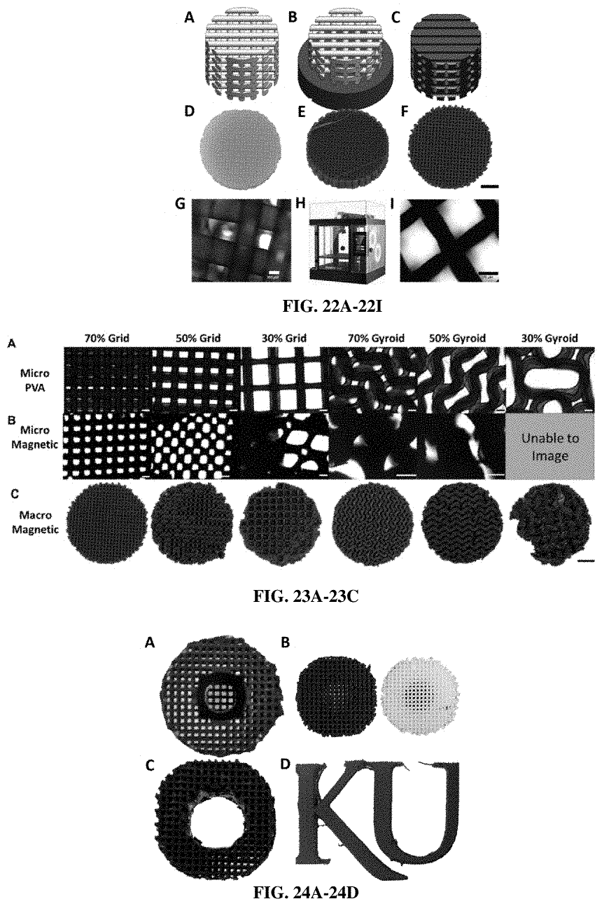

A range of infill volume percentages (30%, 50%, 70%) were also studied to analyze the potential bounds in controlling the porosity, mechanical properties, interface dimensions, and geometries to influence cellular behavior. Microscopic images of 3D printed PVA sacrificial templates were shown in FIG. 23A which exhibited the unique and precisely defined 3D microstructures. The casted magnetic PDMS composites replicate such 3D porous microstructures with excellent precision as demonstrated sequentially in FIG. 23B. FIG. 23C is the overview of resulting 3D magnetic PDMS composites with different porous geometries. We observed the PVA template with 70% infill volume could lead to robust and reproducible fabrication of 3D magnetic PDMS composites regardless of the porous geometries. In contrast, 50% and 30% infill volume both led to a certain degree of defects on both microscale (FIG. 23B) and macroscale (FIG. 23C) levels during the casting process, and 30% infill volume form PVA template showed much more structural and fractal abnormalities for both grid and gyroid constructs, making them unable to develop a fully intact 3D construct, particularly for gyroid-shaped constructs. Thus, these results indicate that the infill volume range in PVA template for the development of reproducible high-volume magnetic PDMS porous constructs is between 50-70%, which casts sufficient PDMS material for maintaining the mechanical strength in support 3D microstructures. Although higher infill volume in PVA template is attainable with larger magnetic-PDMS porous constructs (diameter>1 cm), we are more interested in developing 3D constructs suitable as the insert in 96 well plates for 3D cell culture employed in high throughput drug screening.

Because of the increased distance between strands, the liquid-air surface tension and adhesive forces were not sufficient for inducing capillary action to infill the PVA template volume with magnetic-PDMS precursor at lower infill percentages (<50%). The inability to establish the full porosity range exhibited by Mohanty, S. et al. was likely due to the higher density of the carbonyl iron and PDMS precursor compared to the PDMS only precursor (980 vs. 4420 kg/m³). This result can be described by the equation for capillary action Eq. (1), as the height that the liquid precursor can travel (h) will decrease with both an increase in distance between the strands (r) and density (p) with the assumption that there is little or no change in the liquid-air surface tension (y) and contact angle (θ).

$$h = \frac{2 γ cos θ}{ρ g r} \qquad ( 1 )$$

However, this result is tolerable given higher percentage infill PVA templates lead to higher porous constructs. This is desired for interface and scaffolding in 3D cell culture systems, due to the ability for higher nutrient and waste transport from higher interconnected microchannels. The higher infill PVA templates also allow for casting smaller strand widths of the magnetic porous composites, which is advantageous when considering topographical contact points for controlled alignment of cells in particular tissue sets such as bones, muscles, and nerves. Such tissue sets have been extensively studied in vitro but is usually limited to a means for static control of 3D dynamic cell culture.⁸⁻¹⁰Our developed platform supplies suitable topographical contact points and mass transport for interfacing 3D cell culture with remote-control of dynamic conditions, which could advance deep understanding in a variety of tissue engineering models. Further developments can study the usage of smaller FDM nozzles to explore the ability to fabricate 3D structures with smaller strand gaps to yield both more porous and smaller strand width magnetic porous composites.

Owing to the power of 3D printing, many complicated macroscopic designs can be achieved to cast the 3D magnetic PDMS porous composites with well-defined microstructures and high fidelity. These structural examples were included in FIG. 24A-D, which proves that broad applications can be developed. Small feature and high aspect ratio characteristics were obtained which is illustrated in the 2 mm×4 mm well developed in a cylinder (FIG. 24A) and the ability to form a void space within a cylinder with a reservoir construct (FIG. 24B). Complex structures are also attainable with highly specific features shown in the hollow cylinder (FIG. 24C) and KU logo (FIG. 24D). These presented designs showcase the ability to develop complex structures at both the micro and macro scale that can be translated to the development of organ specific interfaces and scaffolds.

3D Magnetic PDMS Porous Composites Enable the Remote-Controlled Actuation

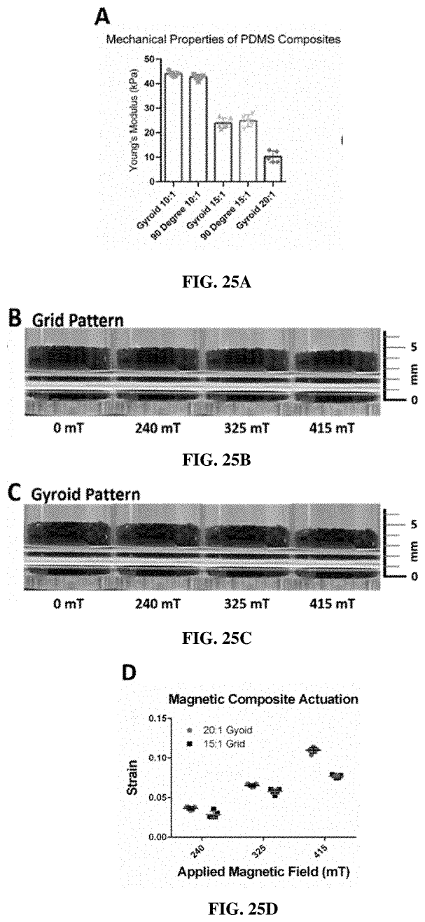

The non-destructive, remotely controlled actuation in the field of tissue engineering is emerging, especially for inducing dynamic mechanical strain which could play a large role in the fate of mesenchymal stem cells^{35,36,48,49} Carbonyl iron microparticles were chosen to be embedded within PDMS given their high magnetization level with a low hysteresis which leads to a strong translational force in the direction of an applied field⁵⁰. When this combinational force is greater than the intrinsic stiffness of the PDMS then this will lead to a deformation of the bulk PDMS, which is why we observed that actuation capacity of our developed 3D magnetic PDMS porous composites is directly correlated to their intrinsic mechanical properties. We were able to control such mechanical properties by altering the stiffness of PDMS composites casted from 70% infill grid and gyroid PVA templates which possessed the high-fidelity in small constructs with higher porosity to increase actuation potential. Three different base-to-cure cross linking ratios (10:1, 15:1, 20:1) were studied to yield less stiff PDMS composites at higher ratios. Strain-dependent uniaxial compression of the magnetic composites was performed to assess the Young's modulus from all the samples shown in FIG. 25A. The compressive data reveals the capacity to tune the mechanical stress of the porous magnetic PDMS composites from 10.17±2.31 to 44.01±1.08 kPa by altering the base to curing ratio from 20:1 to 10:1. There was no statistically significant difference found between the grid and gyroid patterned porous composites with the same base to curing agent ratio, which indicates the consistent fabrication protocols between 3D constructs. This also seems to indicate that the porous composite stiffness is independent from these two infill patterns

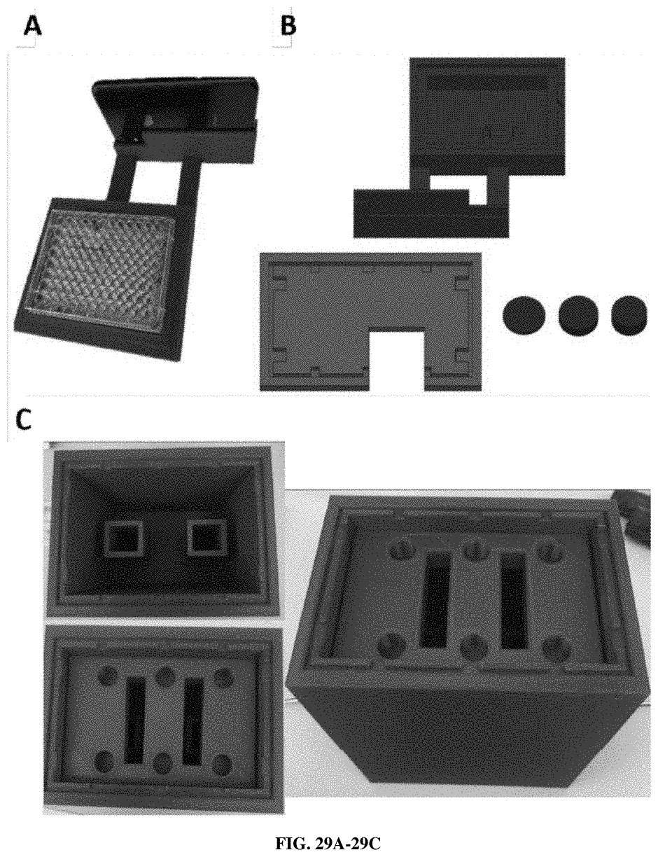

The ability to alter the intrinsic stiffness of the magnetic porous composites should allow for a wide mechanical strain range, with the less stiff composites having the largest strain potential. Thus, we investigated the remotely controlled deformation on the lowest stiffness attainable grid (15:1) and gyroid (20:1) magnetic composites, respectively in FIGS. 25B and 25C. A 3D printed compartmental box was developed to have both a controlled applied magnetic field and reproducible image (FIGS. 29A and 29B). Also, the entire setup can fit very well with the 96 well plate for high-throughput dynamic 3D cell culture, with the smartphone camera for imaging and video recording. Such integrated setup would allow for both qualitative and quantitative magnetically actuated deformation analysis given the control over the position of the camera and magnetic composite along with the applied magnetic field. Both the grid (15:1) and gyroid (20:1) 3D constructs were fabricated to fit 96 well-plate and were submerged in DPBS to mimic actuation under cell culture conditions. The magnetic composites were then placed under applied magnetic fields of 0, 240, 325, and 415 mT. As expected, macrolevel strain of magnetic composites in both grid and gyroid patterns across all the magnetic field levels were actuated, with higher strain levels while increasing applied magnetic fields. Shown in FIG. 25D, the grid construct experiences 2.89 (+/−0.41)%, 5.87 (+/−0.36)%, and 7.71 (+/−0.20)% strain actuation, while the gyroid constructs encountered 3.67 (+/−0.22) %, 6.57 (+/−0.20) %, and 11.00 (+/−0.43) % strain actuation along with increased magnetic field intensities (240, 325, and 415 mT). The higher actuation potential of the gyroid constructs could be correlated to its lower stiffness at a 20:1 ratio, although the magnetic volume fraction of embedded magnetic particles was kept constant in all magnetic-PDMS composite groups.

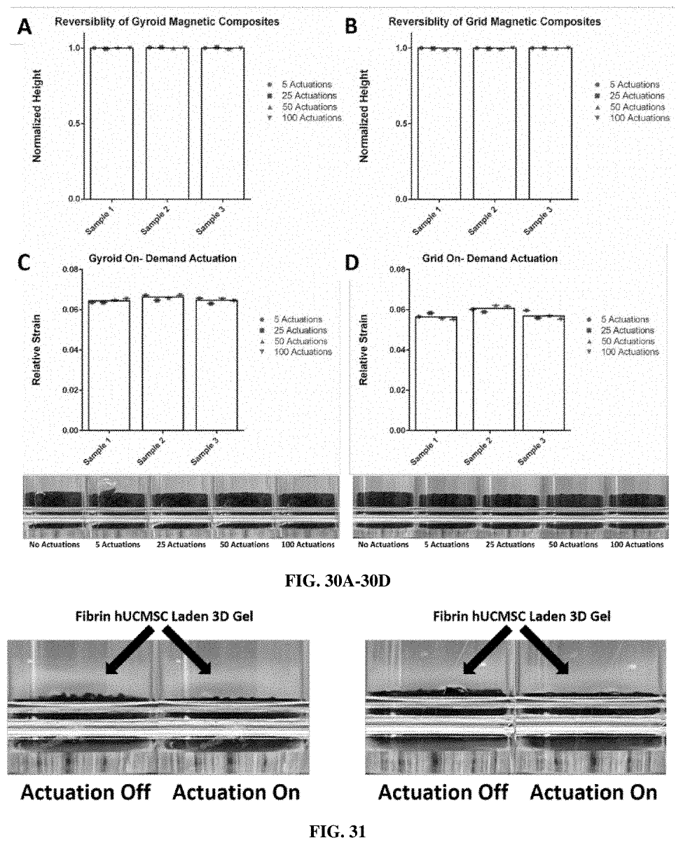

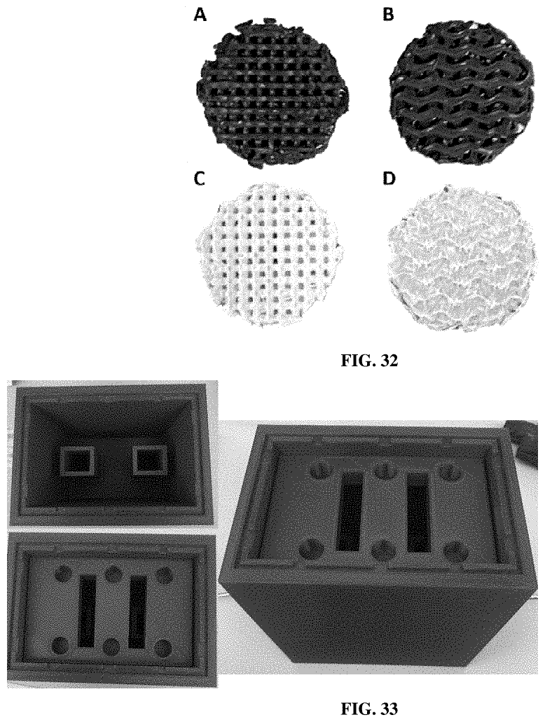

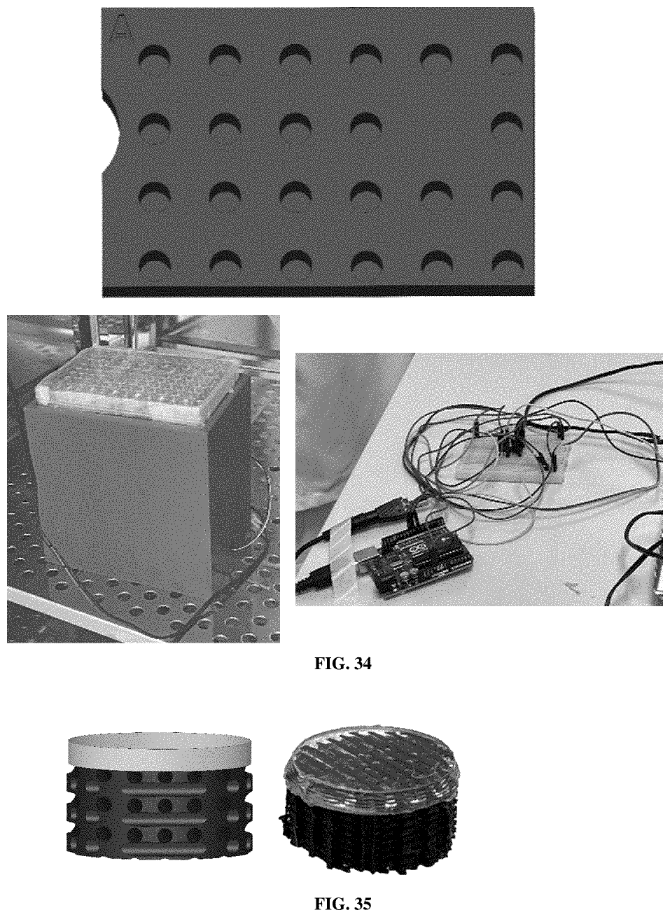

Additional dynamic magnetic composite actuation studies were then evaluated to establish the on-demand and precise actuation capacity of the magnetic porous composites. A dynamic actuation compartmental box was developed to utilize two linear actuators to control the z-directional movement of a stage, encompassing an array of permanent magnetism to control the dynamic magnetic field experienced by a magnetic porous composite (seen in FIG. 29C). The first investigation studied the capability of both a gyroid (20:1) and grid (15:1) to reverse back to its original height after multiple different dynamic actuation cycles (5, 25, 50, and 100) with an applied magnetic field of 325 mT. The results shown in FIGS. 30A and 30B reveal that both the gyroid and grid were able to reverse back to within 0.1% of their original height across all the different dynamic actuation cycle durations. Also, when it came to assessing the precise control of the magnetic composite strain levels after numerous actuation cycle time points, the composites revealed a consistent strain response with only an average standard deviation of 0.0011 and 0.0016, respectively for the gyroid and grid structures strain levels.

Cyclic strain actuation of a gyroid construct at 325 mT can be observed across a range of different frequencies (0.25, 1, and 2 Hz). In addition, hUCMSC laden fibrin gels were polymerized on the top of gyroid (20:1) magnetic composites, that were the same height (3 mm) of the porous composites used for cell viability studies, and actuated with a 325 mT to show the ability for the composites to induce a strain that could be translated to the 3D fibrin gel placed above. FIG. 31 demonstrates that the magnetic composites deform (6.00% and 6.33%) with a cell laden fibrin gel under dynamic magnetic actuation conditions.

With the proposed magnetic porous composite bottom surface interface, the magnetic porous composite will induce a tensile strain utilizing similar design principles utilized by the commercially available Flexcell® Tissue Train® 3D Cell Culture System and other 3D tensile strain platforms⁵¹⁻⁵², which use fixed constraints to anchor the cell culture on select surfaces and have one or more stretchable interface substrates to induce a stretching event. For the particular system studied, the angular surface of the well plate acts as the anchor surface with the PDMS interface acting as the deformable surface that induces a uniaxial stretching event. The height of the 3D fibrin gel interfaced with the magnetic composite can also be modified to control the translatable strain experienced by the 3D gel as a thinner gel will result in a higher strain.

The higher actuation potential of the gyroid constructs could be correlated to its lower stiffness at a 20:1 ratio, although the magnetic volume fraction of embedded magnetic particles was kept constant in all magnetic-PDMS composite groups. The reproducible magnetic porous composite strain levels studied here are comparable with the compression and tensile mechanical stimuli used in controlling the differentiation of MSCs in 3D gels from reported actuation studies (5-15% strain)^{19,53,54}, which indicates the feasibility for using our developed 3D magnetic PDMS composites as dynamic interface to induce physiological mechanical actuation.

3D Porous Ferromagnetic Interface Enables the 3D Dynamic Culture of hMSCs

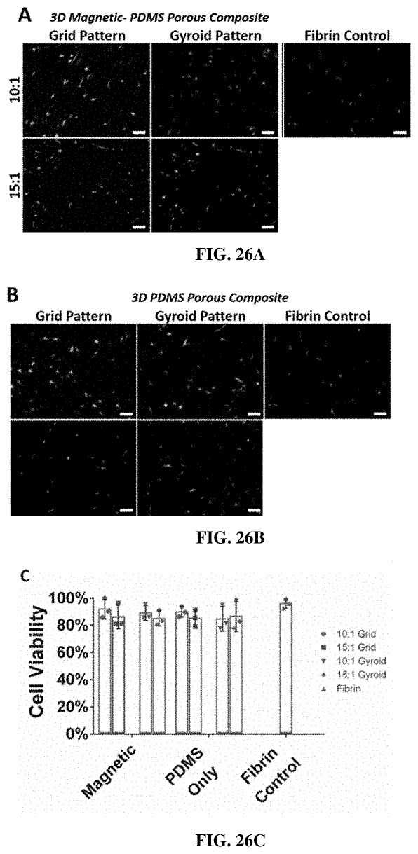

Although micropatterning has been shown to control the alignment of hMSCs in 2D culture,^{45,55,56} the 3D patterned dynamic interfaces have not been well studied due to the lack of capable dynamic micropattern platforms which is critically needed as an improved 3D culture system in mimicking in vivo MSCs cellular behavior. Our developed 3D magnetic PDMS porous composites are desired as both scaffolding and interface materials for precise remote-control of the dynamic mechanical environment in a 3D cell culture system. FIG. 25 already demonstrated the potential using as the interface material by offering seamlessly comparable mechanical actuation to the 3D cell culture scaffolding environment. Herein, we further studied the biocompatibility and cell viability for interfacing with 3D cell culture system. The studies were implemented by placing a 3D cell-laden gel on the top surface of the magnetic porous materials to assess any influences on hampering the cell growth behavior due to the porous surface contacts or magnetic particle contaminations. Different porous construct groups (FIG. 29), in terms of magnetic loading weight fractions to PDMS, infill patterns, and PDMS base to curing agent ratios, were used for cell viability studies shown in FIG. 26. The cell-laden fibrin gel was used as the control group as the natural 3D scaffolding material.

Patient derived human umbilical cord stem cells were chosen as the cell source owing to their sensitive response to various mechanical loading in culturing environment and be able to differentiate into adipose, bone, tendon, muscle, and cartilage linages depending on both the intrinsic and extrinsic mechanical environments provided.^{6,53,54} Fibrin gels with final concentrations of 2.5 U/mL thrombin and 11.25 mg/mL fibrinogen were polymerized on the top of magnetic composites and PDMS porous constructs. The live-dead two-color cell viability assay was performed after four days of cell culture. High cell viability (>84%) was observed across all the experimental interface groups and were comparable to the positive fibrin control group (FIG. 26C). Most importantly, both of the 10:1 magnetic porous composite groups exhibited cell viabilities above 89%. The results indicate that no carbonyl iron-contaminates or detrimental effects due to the interface pattern were observed revealing a biocompatible, 3D porous ferromagnetic interface with remotely controlled, on-demand dynamic strain actuation function. Further studies will consist of observing the cellular behavior response due to both the mechanical actuation of the magnetic porous actuation and the anisotropic interface that is presented to the surface of the 3D cell culture.

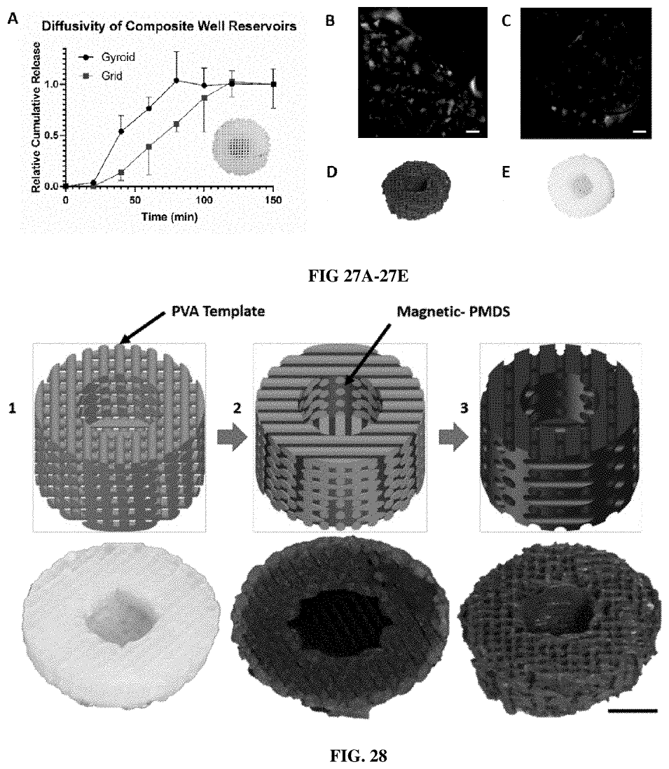

We also implemented the specific 3D microstructure designs which is imperative for improving the waste and nutrient mass transport and exchange through the interconnected networks of the 3D porous ferromagnetic constructs. We introduced a reservoir interface design which could provide a dynamic micromechanical environment to all surfaces of a 3D cell-laden gel for highly controlled actuation. Both grid and gyroid patterned reservoirs were designed with 3 mm thick, and porous walls surrounding a 5×5 mm reservoir space for housing an alginate sol gel (5%, w/w). Mass transport analysis was then determined by the ability for fluid to move from the gel through the exterior porous walls to the bulk fluid. The mass transport out of reservoir composite structures was quantified by incorporating aniline blue dye into the gels and measuring the absorbance of the bulk fluid at different time points shown in FIG. 27A. As expected, an initial lag phase was observed as the dye in the gel needs to both dissolute and transport through the interconnected network of the reservoir walls to the bulk fluid. The gyroid structures emerged as the best micropattern design for waste and nutrient transport by showing a faster mean release profile than that of the grid pattern in FIG. 27A. Because of the minimal surface designed in gyroid structures, the higher permeability is expected given the single connected and infinite domains. As a result, optimal interconnectivity is achieved given there are no sealed cavities in the structure⁴⁶. Although there are different initial penetration profiles, both gyroid and grid designs showed the capability for usage as interface and scaffold materials given their suitable interconnected networks for improving the mass transport. It is worth mentioning that fluid penetration through PDMS porous structure is very challenging at the initial penetration phase due to the limited wettability. However, mechanical actuation could improve this process to ensure the good fluid penetration before the loading of the gels. The collected release data was standardized to account for the gel loading variability. Through the compression based actuation, any unwanted pressure waves can be avoided for seamless transport¹⁹. Most importantly, the 3D sacrificial template allows for casting versatile pore shapes and precisely controlling interconnected porosity which could substantially improve the permeability and mass transport within the 3D constructs.

A 3D cell culture and viability study were further performed in this 3D well construct using human derived adipose mesenchymal stem cells (hADMSCs) which is a different type of mesenchymal stem cell source and more available than hUCMSCs used in FIG. 26. The structure consisted of a 4 mm diameter and 2 mm tall well, which encompassed the 3D gel fibrin gel, and had 3 mm thick, porous walls and the top of the 3D fibrin gel was exposed to the bulk fluid. A live-dead two-color cell viability assay was performed after five days of cell culture to further demonstrate the suitable cell viability of the 3D cell cultures in the well constructs due to excellent waste and nutrient transport. There is no noticeable difference in the cell viability amongst the cells cultured in the magnetic (FIG. 27B) or PDMS-only constructs (FIG. 27C). Thus, the high cell viability under the well construct culture conditions indicate the feasibility and potential for dynamic 3D cell culture.

### CONCLUSION

Magnetic stimuli responsive materials have been emerging as the next generation of dynamic cell culture platforms. These materials allow for fast, highly tunable actuation at both the macroscopic and microscopic scale without any external pressure systems and tethered electronics, leading to the ease of integration into high-throughput systems such as 96 cell culture well plates and microfluidic chips. However, current existing bioreactor systems using such magnetic responsive materials are still lacking the precision in microenvironment control, or in low throughput and bulky size. In this research, we overcame such limitations by developing a novel magnetic elastomer cell culture platform and using a porous magnetic PDMS composite with the full tunability of microstructures. The fabrication of the 3D magnetic PDMS composites are simple and straightforward with high-fidelity control of microlevel and macrolevel structures, which can reproduce strain levels suited in 3D stem cell culture and differentiation. The porous magnetic PDMS composite also demonstrated biocompatibility given the high cell viability experienced for 3D culturing both hUCMSC and hADMSCs. The high interconnected porosity of the porous structure also supports the development of a multiple surface contact design for more complex mechanical actuation given improved fluid permeability and mass transport. Our results reveal a promising biocompatible, remotely controlled magnetic PDMS composite interface material with full control over the porosity, microlevel and macrolevel structure, mechanical properties, and dynamic strain for tunable dynamic cell culture studies at the scale. For future studies, multiple embodiment designs will be established to look at both tensile and compression actuation and the ability to control the differentiation of hUCMSCs. The magnetic actuation device will be developed to remotely control the strain levels of an array of porous magnetic-PDMS composites in a cell culture plate to induce a well-defined dynamic mechanical environment in high throughput employed in drug screening.

### REFERENCES

1. Song, Y.; Soto, J.; Chen, B.; Yang, L.; Li, S., Cell engineering: Biophysical regulation of the nucleus. Biomaterials 2020, 234.
2. Vining, K. H.; Mooney, D. J., Mechanical forces direct stem cell behaviour in development and regeneration. Nature Reviews Molecular Cell Biology 2017, 18 (12), 728-742.
3. Sun, Z.; Guo, S. S.; Fässler, R., Integrin-mediated mechanotransduction. Journal of Cell Biology 2016, 215 (4), 445-456.
4. Martino, F.; Perestrelo, A. R.; Vinarský, V.; Pagliari, S.; Forte, G., Cellular Mechanotransduction: From Tension to Function. Front Physiol 2018, 9.
5. Chowdhury, F.; Na, S.; Li, D.; Poh, Y.-C.; Tanaka, T. S.; Wang, F.; Wang, N., Material properties of the cell dictate stress-induced spreading and differentiation in embryonic stem cells. Nature Materials 2009, 9 (1), 82-88.
6. Engler, A. J.; Sen, S.; Sweeney, H. L.; Discher, D. E., Matrix Elasticity Directs Stem Cell Lineage Specification. Cell 2006, 126 (4), 677-689.
7. Gilbert, P. M.; Havenstrite, K. L.; Magnusson, K. E. G.; Sacco, A.; Leonardi, N. A.; Kraft, P.; Nguyen, N. K.; Thrun, S.; Lutolf, M. P.; Blau, H. M., Substrate Elasticity Regulates Skeletal Muscle Stem Cell Self-Renewal in Culture. Science 2010, 329 (5995), 1078-1081.
8. Kilian, K. A.; Bugarija, B.; Lahn, B. T.; Mrksich, M., Geometric cues for directing the differentiation of mesenchymal stem cells. Proceedings of the National Academy of Sciences 2010, 107 (11), 4872-4877.
9. McMurray, R. J.; Gadegaard, N.; Tsimbouri, P. M.; Burgess, K. V.; McNamara, L. E.; Tare, R.; Murawski, K.; Kingham, E.; Oreffo, R. O. C.; Dalby, M. J., Nanoscale surfaces for the long-term maintenance of mesenchymal stem cell phenotype and multipotency. Nature Materials 2011, 10 (8), 637-644.
10. Nikkhah, M.; Edalat, F.; Manoucheri, S.; Khademhosseini, A., Engineering microscale topographies to control the cell-substrate interface. Biomaterials 2012, 33 (21), 5230-5246.
11. Steward, A. J.; Thorpe, S. D.; Vinardell, T.; Buckley, C. T.; Wagner, D. R.; Kelly, D. J., Cell-matrix interactions regulate mesenchymal stem cell response to hydrostatic pressure. Acta Biomaterialia 2012, 8 (6), 2153-2159.
12. Ju, W. K.; Kim, K. Y.; Lindsey, J. D.; Angert, M.; Patel, A.; Scott, R. T.; Liu, Q.; Crowston, J. G.; Ellisman, M. H.; Perkins, G. A.; Weinreb, R. N., Elevated hydrostatic pressure triggers release of OPA1 and cytochrome C, and induces apoptotic cell death in differentiated RGC-5 cells. Mol Vis 2009, 15 (12-13), 120-134.
13. Barron, M. J.; Goldman, J.; Tsai, C.-J.; Donahue, S. W., Perfusion Flow Enhances Osteogenic Gene Expression and the Infiltration of Osteoblasts and Endothelial Cells into Three-Dimensional Calcium Phosphate Scaffolds. International Journal of Biomaterials 2012, 2012, 1-10.
14. Bancroft, G. N.; Sikavitsas, V. I.; van den Dolder, J.; Sheffield, T. L.; Ambrose, C. G.; Jansen, J. A.; Mikos, A. G., Fluid flow increases mineralized matrix deposition in 3D perfusion culture of marrow stromal osteoblasts in a dose-dependent manner. Proceedings of the National Academy of Sciences 2002, 99 (20), 12600-12605.
15. Baeyens, N.; Nicoli, S.; Coon, B. G.; Ross, T. D.; Van den Dries, K.; Han, J.; Lauridsen, H. M.; Mejean, C. O.; Eichmann, A.; Thomas, J.-L.; Humphrey, J. D.; Schwartz, M. A., Vascular remodeling is governed by a VEGFR3-dependent fluid shear stress set point. eLife 2015, 4.
16. Chen, P.-Y.; Qin, L.; Baeyens, N.; Li, G.; Afolabi, T.; Budatha, M.; Tellides, G.; Schwartz, M. A.; Simons, M., Endothelial-to-mesenchymal transition drives atherosclerosis progression. Journal of Clinical Investigation 2015, 125 (12), 4514-4528.
17. Thorpe, S. D.; Buckley, C. T.; Vinardell, T.; O'Brien, F. J.; Campbell, V. A.; Kelly, D. J., The Response of Bone Marrow-Derived Mesenchymal Stem Cells to Dynamic Compression Following TGF-β3 Induced Chondrogenic Differentiation. Annals of Biomedical Engineering 2010, 38 (9), 2896-2909.
18. McKee, C.; Hong, Y.; Yao, D.; Chaudhry, G. R., Compression Induced Chondrogenic Differentiation of Embryonic Stem Cells in Three-Dimensional Polydimethylsiloxane Scaffolds. Tissue Engineering Part A 2017, 23 (9-10), 426-435.
19. Occhetta, P.; Mainardi, A.; Votta, E.; Vallmajo-Martin, Q.; Ehrbar, M.; Martin, I.; Barbero, A.; Rasponi, M., Hyperphysiological compression of articular cartilage induces an osteoarthritic phenotype in a cartilage-on-a-chip model. Nature Biomedical Engineering 2019, 3 (7), 545-557.
20. Guo, T.; Yu, L.; Lim, C. G.; Goodley, A. S.; Xiao, X.; Placone, J. K.; Ferlin, K. M.; Nguyen, B.-N. B.; Hsieh, A. H.; Fisher, J. P., Effect of Dynamic Culture and Periodic Compression on Human Mesenchymal Stem Cell Proliferation and Chondrogenesis. Annals of Biomedical Engineering 2015, 44 (7), 2103-2113.
21. Sumanasinghe, R. D.; Bernacki, S. H.; Loboa, E. G., Osteogenic Differentiation of Human Mesenchymal Stem Cells in Collagen Matrices: Effect of Uniaxial Cyclic Tensile Strain on Bone Morphogenetic Protein (BMP-2) mRNA Expression. Tissue Engineering 2006, 12 (12), 3459-3465.
22. Lohberger, B.; Kaltenegger, H.; Stuendl, N.; Payer, M.; Rinner, B.; Leithner, A., Effect of Cyclic Mechanical Stimulation on the Expression of Osteogenesis Genes in Human Intraoral Mesenchymal Stromal and Progenitor Cells. BioMed Research International 2014, 2014, 1-10.
23. Charoenpanich, A.; Wall, M. E.; Tucker, C. J.; Andrews, D. M. K.; Lalush, D. S.; Dirschl, D. R.; Loboa, E. G., Cyclic Tensile Strain Enhances Osteogenesis and Angiogenesis in Mesenchymal Stem Cells from Osteoporotic Donors. Tissue Engineering Part A 2014, 20 (1-2), 67-78.
24. Marycz, K.; Lewandowski, D.; Tomaszewski, K. A.; Henry, B. M.; Golec, E. B.; Maredziak, M., Low-frequency, low-magnitude vibrations (LFLM) enhances chondrogenic differentiation potential of human adipose derived mesenchymal stromal stem cells (hASCs). PeerJ 2016, 4.
25. Campsie, P.; Childs, P. G.; Robertson, S. N.; Cameron, K.; Hough, J.; Salmeron-Sanchez, M.; Tsimbouri, P. M.; Vichare, P.; Dalby, M. J.; Reid, S., Design, construction and characterisation of a novel nanovibrational bioreactor and cultureware for osteogenesis. Sci Rep 2019, 9 (1).
26. Kim, I. S.; Song, Y. M.; Lee, B.; Hwang, S. J., Human Mesenchymal Stromal Cells are Mechanosensitive to Vibration Stimuli. Journal of Dental Research 2012, 91 (12), 1135-1140.
27. Zhao, J.; Griffin, M.; Cal, J.; Li, S.; Bulter, P. E. M.; Kalaskar, D. M., Bioreactors for tissue engineering: An update. Biochemical Engineering Journal 2016, 109, 268-281.
28. Fowler, S.; Chen, W. L. K.; Duignan, D. B.; Gupta, A.; Hariparsad, N.; Kenny, J. R.; Lai, W. G.; Liras, J.; Phillips, J. A.; Gan, J., Microphysiological systems for ADME-related applications: current status and recommendations for system development and characterization. Lab on a Chip 2020, 20 (3), 446-467.
29. Sart, S.; Agathos, S. N.; Li, Y.; Ma, T., Regulation of mesenchymal stem cell 3D microenvironment: From macro to microfluidic bioreactors. Biotechnol J 2016,11 (1), 43-57.
30. Huh, D.; Leslie, D. C.; Matthews, B. D.; Fraser, J. P.; Jurek, S.; Hamilton, G. A.; Thorneloe, K. S.; McAlexander, M. A.; Ingber, D. E., A Human Disease Model of Drug Toxicity—Induced Pulmonary Edema in a Lung-on-a-Chip Microdevice. Science Translational Medicine 2012, 4 (159), 159ra147-159ra147.
31. Marsano, A.; Conficconi, C.; Lemme, M.; Occhetta, P.; Gaudiello, E.; Votta, E.; Cerino, G.; Redaelli, A.; Rasponi, M., Beating heart on a chip: a novel microfluidic platform to generate functional 3D cardiac microtissues. Lab on a Chip 2016, 16 (3), 599-610.
32. Costa, J.; Ghilardi, M.; Mamone, V.; Ferrari, V.; Busfield, J. J. C.; Ahluwalia, A.; Carpi, F., Bioreactor With Electrically Deformable Curved Membranes for Mechanical Stimulation of Cell Cultures. Front Bioeng Biotechnol 2020, 8.
33. Poulin, A.; Imboden, M.; Sorba, F.; Grazioli, S.; Martin-Olmos, C.; Rosset, S.; Shea, H., An ultra-fast mechanically active cell culture substrate. Sci Rep 2018, 8 (1).
34. Costa-Rodrigues, J.; Mayer, M.; Rabindranath, R.; Borner, J.; Horner, E.; Bentz, A.; Salgado, J.; Han, H.; Bose, H.; Probst, J.; Shamonin, M.; Monkman, G. J.; Schlunck, G., Ultra-Soft PDMS-Based Magnetoactive Elastomers as Dynamic Cell Culture Substrata. PLoS ONE 2013, 8 (10).
35. Bidan, C. M.; Fratzl, M.; Coullomb, A.; Moreau, P.; Lombard, A. H.; Wang, I.; Balland, M.; Boudou, T.; Dempsey, N. M.; Devillers, T.; Dupont, A., Magneto-active substrates for local mechanical stimulation of living cells. Sci Rep 2018, 8 (1).
36. Yang, J.-W.; Chen, Y.-W.; Ho, P.-Y.; Jiang, L.; Hsieh, K. Y.; Cheng, S.-J.; Lin, K.-C.; Lu, H.-E.; Chiu, H.-Y.; Lin, S.-F.; Chen, G.-Y., The Development of Controllable Magnetic Driven Microphysiological System. Frontiers in Cell and Developmental Biology 2019, 7.
37. Kojima, T.; Husari, A.; Dieterle, M. P.; Fontaine, S.; Prucker, O.; Tomakidi, P.; Rae, J., PnBA/PDMAA-Based Iron-Loaded Micropillars Allow for Discrete Cell Adhesion and Analysis of Actuation-Related Molecular Responses. Advanced Materials Interfaces 2020, 7 (7).
38. Kim, Y.; Yuk, H.; Zhao, R.; Chester, S. A.; Zhao, X., Printing ferromagnetic domains for untethered fast-transforming soft materials. Nature 2018, 558 (7709), 274-279.
39. Lui, Y. S.; Sow, W. T.; Tan, L. P.; Wu, Y.; Lai, Y.; Li, H., 4D printing and stimuli-responsive materials in biomedical aspects. Acta Biomaterialia 2019, 92, 19-36.
40. Jackson, J. A.; Messner, M. C.; Dudukovic, N. A.; Smith, W. L.; Bekker, L.; Moran, B.; Golobic, A. M.; Pascall, A. J.; Duoss, E. B.; Loh, K. J.; Spadaccini, C. M., Field responsive mechanical metamaterials. Science Advances 2018, 4 (12).
41. Neves, N. M.; Palchesko, R. N.; Zhang, L.; Sun, Y.; Feinberg, A. W., Development of Polydimethylsiloxane Substrates with Tunable Elastic Modulus to Study Cell Mechanobiology in Muscle and Nerve. PLoS ONE 2012, 7 (12).
42. Shi, J.; Zhang, H.; Jackson, J.; Shademani, A.; Chiao, M., A robust and refillable magnetic sponge capsule for remotely triggered drug release. Journal of Materials Chemistry B 2016, 4 (46), 7415-7422.
43. Shademani, A.; Zhang, H.; Jackson, J. K.; Chiao, M., Active Regulation of On-Demand Drug Delivery by Magnetically Triggerable Microspouters. Adv Funct Mater 2017, 27 (6).
44. Mohanty, S.; Larsen, L. B.; Trifol, J.; Szabo, P.; Burri, H. V. R.; Canali, C.; Dufva, M.; Emnéus, J.; Wolff, A., Fabrication of scalable and structured tissue engineering scaffolds using water dissolvable sacrificial 3D printed moulds. Materials Science and Engineering: C 2015, 55, 569-578.
45. Miao, S.; Cui, H.; Esworthy, T.; Mahadik, B.; Lee, S. j.; Zhou, X.; Hann, S. Y.; Fisher, J. P.; Zhang, L. G., 4D Self-Morphing Culture Substrate for Modulating Cell Differentiation. Advanced Science 2020, 7 (6).
46. Kapfer, S. C.; Hyde, S. T.; Mecke, K.; Arns, C. H.; Schroder-Turk, G. E., Minimal surface scaffold designs for tissue engineering. Biomaterials 2011, 32 (29), 6875-6882.
47. Montazerian, H.; Mohamed, M. G. A.; Montazeri, M. M.; Kheiri, S.; Milani, A. S.; Kim, K.; Hoorfar, M., Permeability and mechanical properties of gradient porous PDMS scaffolds fabricated by 3D-printed sacrificial templates designed with minimal surfaces. Acta Biomaterialia 2019, 96, 149-160.
48. Du, V.; Luciani, N.; Richard, S.; Mary, G.; Gay, C.; Mazuel, F.; Reffay, M.; Menasché, P.; Agbulut, O.; Wilhelm, C., A 3D magnetic tissue stretcher for remote mechanical control of embryonic stem cell differentiation. Nature Communications 2017, 8 (1).
49. Enríquez, Á.; Libring, S.; Field, T. C.; Jimenez, J.; Lee, T.; Park, H.; Satoski, D.; Wendt, M. K.; Calve, S.; Tepole, A. B.; Solorio, L.; Lee, H., High-Throughput Magnetic Actuation Platform for Evaluating the Effect of Mechanical Force on 3D Tumor Microenvironment. Adv Funct Mater 2020, 31 (1).
50. Roh, S.; Okello, L. B.; Golbasi, N.; Hankwitz, J. P.; Liu, J. A. C.; Tracy, J. B.; Velev, O. D., 3D-Printed Silicone Soft Architectures with Programmed Magneto-Capillary Reconfiguration. Advanced Materials Technologies 2019, 4 (4).
51. Liu, H.; MacQueen, L. A.; Usprech, J. F.; Maleki, H.; Sider, K. L.; Doyle, M. G.; Sun, Y.; Simmons, C. A., Microdevice arrays with strain sensors for 3D mechanical stimulation and monitoring of engineered tissues. Biomaterials 2018, 172, 30-40.
52. Kamble, H.; Vadivelu, R.; Barton, M.; Boriachek, K.; Munaz, A.; Park, S.; Shiddiky, M.; Nguyen, N.-T., An Electromagnetically Actuated Double-Sided Cell-Stretching Device for Mechanobiology Research. Micromachines 2017, 8 (8).
53. McClarren, B.; Olabisi, R., Strain and Vibration in Mesenchymal Stem Cells. International Journal of Biomaterials 2018, 2018, 1-13.
54. Choi, J. R.; Yong, K. W.; Choi, J. Y., Effects of mechanical loading on human mesenchymal stem cells for cartilage tissue engineering. Journal of Cellular Physiology 2018, 233 (3), 1913-1928.
55. Li, S.; Kuddannaya, S.; Chuah, Y. J.; Bao, J.; Zhang, Y.; Wang, D., Combined effects of multi-scale topographical cues on stable cell sheet formation and differentiation of mesenchymal stem cells. Biomaterials Science 2017, 5 (10), 2056-2067.
56. Tay, C. Y.; Yu, H.; Pal, M.; Leong, W. S.; Tan, N. S.; Ng, K. W.; Leong, D. T.; Tan, L. P., Micropatterned matrix directs differentiation of human mesenchymal stem cells towards myocardial lineage. Experimental Cell Research 2010, 316 (7), 1159-1168.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

What is claimed is:

1. A system for cell culture comprising:
a vessel;
a porous, magnetic, elastomeric construct sized to be positioned within the vessel; and
a magnet configured to apply a magnetic field within the construct effective to deform the construct.

2. The system of claim 1, wherein the vessel is substantially cylindrical and comprises a bottom and a side wall.

3. The system of claim 1, wherein the vessel comprises a well of multiwell plate having a circular bottom surface and a side wall.

4. The system of claim 1, wherein the construct has a substantially cylindrical shape.

5. The system of claim 4, wherein the diameter of the construct is from 60% to 99% of the inner diameter of the well.

6. The system of claim 1, wherein the porous, magnetic, elastomeric construct is formed from a composite comprising a biocompatible elastomer and a population of magnetic particles dispersed within the biocompatible elastomer.

7. The system of claim 6, wherein the biocompatible elastomer comprises a acrylonitrile butadiene styrene (ABS), polyphenylene sulfide (PPS), poly(meth)acrylate, polyphenylsulfone (PPSU), cyclic olefin copolymer (COC), polyetheretherketone (PEEK), polyurethane (PU), polyetherimide (PEI), polyphenylene ether (PPE), polycarbonate (PC), poly(ethyleneterephthalate glycol) (PETG), polysiloxane, or any combination thereof.

8. The system of claim 7, wherein the biocompatible elastomer comprises a polysiloxane, such as polydimethylsiloxane (PDMS).

9. The system of claim 6, wherein the magnetic particles comprise iron, cobalt, zinc, cadmium, nickel, gadolinium, chromium, copper, gold, silver, platinum, manganese, metal oxide, or an alloy thereof.

10. The system of claim 6, wherein the magnetic particles are present in an amount of from 0.5% to 20% by weight, based on the total weight of the construct.

11. The system of claim 1, wherein the porous, magnetic, elastomeric construct has a porosity of from 10% to 50%.

12. The system of claim 1, wherein the porous, magnetic, elastomeric construct is formed by an additive manufacturing process.

13. The system of claim 1, wherein the magnet comprises a permanent magnet.

14. The system of claim 1, wherein the magnet comprises an electromagnet.

15. The system of claim 1, wherein the system further comprises a porous, non-magnetic construct sized to be positioned within the vessel.

16. The system of claim 1, wherein the system further comprises a 3D cell culture matrix sized to be positioned within the vessel.

17. The system of claim 16, wherein the 3D cell culture matrix comprises a population of cells seeded within a degradable polymer matrix.

18. The system of claim 17, wherein the polymer matrix comprises alginate, chitosan, agarose, fibrin, collagen, hyaluronic acid, a polyhydroxyalkanoate, a polyester, a polyalkylene oxide, a copolymer thereof, or a blend thereof.

19. The system of claim 1, wherein the porous, magnetic, elastomeric construct is operatively configured to apply nanovibration, strain, or a combination thereof to a population of cells within the vessel.

20. A method for remote responsive control of dynamic cell culture, the method comprising:
operatively positioning a porous, magnetic, elastomeric construct in proximity to a 3D cell culture matrix; and
applying a magnetic field to the porous, magnetic, elastomeric construct.
